(19) United States
(12) Patent Application Publication
(10) Pub. No.: US 2025/0188194 A1
(43) Pub. Date: Jun. 12, 2025

(54) Method of treatment

(71) Applicants: Nucleus Therapeutics Pty Ltd, South Melbourne, Victoria (AU); Yale University, New Haven, CT (US)

(72) Inventors: James A. CAMPBELL, South Melbourne, Victoria (AU); Valentina DUBLJEVIC, South Melbourne, Victoria (AU); James E. HANSEN, New Haven, CT (US); Xiaoyong CHEN, New Haven, CT (US); Benedette J. CUFFARI, New Haven, CT (US); Anupama SHIRALI, New Haven, CT (US); Jiangbing ZHOU, New Haven, CT (US)

(21) Appl. No.: 18/686,375

(22) PCT Filed: Aug. 30, 2022
(86) PCT No.: PCT/US2022/075639

§ 371 Date: Feb. 23, 2024

Related U.S. Application Data
(60) Provisional application No. 63/238,657, filed on Aug. 30, 2021.
(60) Provisional application No. 63/324,784, filed on Mar. 29, 2022.

(51) Int. Cl. C07K 16/44; A61K 39/00; A61P 37/00

(52) U.S. Cl. CPC C07K 16/44; A61P 37/00; A61K 2039/505; C07K 2317/24

(57) ABSTRACT

The present disclosure relates to therapeutic and pro-phylactic applications of cell penetrating, anti-DNA binding proteins, in particular in the context of inflammatory disease and complications arising from the same.

### FIELD OF THE INVENTION

The present disclosure relates to therapeutic and prophylactic applications of cell penetrating, anti-DNA binding proteins, particularly in the context of inflammatory disease and complications arising from the same.

### BACKGROUND OF THE INVENTION

Activated neutrophils form neutrophil extracellular traps (NETs) that contribute to immune response and the pathophysiology of inflammatory and autoimmune diseases. Chromatin decondensation and citrullination of histones are important events in NETosis, and the DNA damage response contributes to this process. NETs facilitate innate immunity and contribute to multiple disease processes including inflammatory diseases such as acute respiratory distress syndrome and COVID-19, thrombotic disorders, autoimmunity, cystic fibrosis, and potentially to malignancy. Mechanisms of NET-mediated pathophysiology range from damaging effects on epithelial and endothelial cells to promotion of inflammation and autoimmune response through DNA and autoantigen release. Methods to modulate NET formation are of clinical interest, and systemic administration of DNase I or inhibitors of histone citrullination to promote dissolution of NET-DNA or prevent NET formation has been reported. Despite advances in understanding how NETs are formed, effective therapeutic interventions targeting NETosis have yet to emerge.

Accordingly, new compositions and methods for inhibiting NETosis and treating associated pathologies are required.

### SUMMARY OF THE INVENTION

It has been discovered that nuclear-penetrating anti-DNA binding proteins such as autoantibodies and derivatives of the same such as DX1 inhibit both NOX-dependent and independent DNA release and NET formation. It has also been identified that the autoantibodies do not interfere with citrullination of histone H3 but cause its retention within cells. These findings establish the concept of nuclear-penetrating anti-DNA binding proteins as modulators of neutrophil biology with potential for use in strategies to suppress NETosis and, therefore treat and/or inhibit development of inflammatory disease.

Accordingly, in an example, the present disclosure encompasses a method of treating an inflammatory disease in a subject, the method comprising administering to the subject an effective amount of a cell penetrating, anti-DNA binding protein, wherein the cell penetrating, anti-DNA binding protein inhibits Neutrophil Extracellular Trap (NET) formation under culture conditions.

Prophylactic applications are also contemplated. Accordingly, in another example, the present disclosure encompasses a method of inhibiting Neutrophil Extracellular Trap (NET) formation in a subject, the method comprising administering to the subject an effective amount of a cell penetrating, anti-DNA binding protein, wherein the cell penetrating, anti-DNA binding protein inhibits NET formation under culture conditions. In an example, the cell penetrating, anti-DNA binding protein inhibits

NOX-dependent NET formation. In an example, the cell penetrating, anti-DNA binding protein inhibits NOX-independent NET formation. In another example, the cell penetrating, anti-DNA binding protein inhibits both NOX-dependent and NOX-independent NET formation. In another example, the cell penetrating, anti-DNA binding protein does not affect citrullination of histones.

In an example, methods of the disclosure are performed on subjects with an inflammatory disease.

In an example, the administered cell penetrating, anti-DNA binding protein is an autoantibody derived from a subject or an animal with an autoimmune disease, preferably wherein the autoantibody is derived from a subject with systemic lupus erythematous, or an animal model thereof. In an example, the autoantibody is 3E10 or a humanised form thereof.

In another example, the binding protein comprises a V_{H} having a CDR1 as shown in SEQ ID NO: 1, a CDR2 as shown in SEQ ID NO: 2, a CDR3 as shown in SEQ ID NO: 3 and a V_{L} having a CDR 1 as shown in SEQ ID NO: 4, a CDR2 as shown in SEQ ID NO: 5 and a CDR3 as shown in SEQ ID NO: 6 or a humanized form thereof. In another example, the binding protein comprises a V_{H} which comprises an amino acid sequence as shown in SEQ ID NO: 7 and a V_{L} as shown in SEQ ID NO: 8 or a humanized form thereof. In an example, the humanized form thereof comprises a V_{H} having a CDR1 as shown in SEQ ID NO: 9 or SEQ ID NO: 10, a CDR2 as shown in SEQ ID NO: 11 or SEQ ID NO: 12, a CDR3 as shown in SEQ ID NO: 13 and a V_{L} having a CDR1 as shown in SEQ ID NO: 14 or SEQ ID NO: 15, a CDR2 as shown in SEQ ID NO: 16 and a CDR3 as shown in SEQ ID NO: 17 or a variant thereof that competes for binding to DNA with an antibody which comprises a V_{H} having a CDR1 as shown in SEQ ID NO: 9 or SEQ ID NO: 10, a CDR2 as shown in SEQ ID NO: 11 or SEQ ID NO: 12, a CDR3 as shown in SEQ ID NO: 13 and a V_{L} having a CDR1 as shown in SEQ ID NO: 14 or SEQ ID NO: 15, a CDR2 as shown in SEQ ID NO: 16 and a CDR3 as shown in SEQ ID NO: 17. In another example, the humanized form comprises a V_{H} which comprises an amino acid sequence as shown in any one of SEQ ID NOs: 18-21 and a V_{L} as shown in any one of SEQ ID NOs: 22-25 or a variant thereof that competes for binding to DNA with an antibody which comprises a VH which comprises an amino acid sequence as shown in any one of SEQ ID NOs: 18-21 and a VL as shown in any one of SEQ ID NOs: 22-25.

In an example, the binding protein is:
(i) a single chain Fv fragment (scFv);
(ii) a dimeric scFv (di-scFv);
(iii) a trimeric scFv (tri-scFv);
(iv) any one of (i), (ii) or (iii) linked to a constant region of an antibody, Fc or a heavy chain constant domain CH2 and/or CH3
(v) a diabody;
(vi) a triabody;
(vii) a tetrabody;
(viii) a Fab;
(ix) a F(ab′)2;
(x) a Fv;
(xi) any one of (v) to (x) linked to a constant region of an antibody, Fc or a heavy chain constant domain CH2 and/or CH3; or,
(viii) an intact antibody.

In another example, the binding protein is nuclear penetrating. In an example, the nuclear penetrating binding protein inhibits release of DNA from cell nuclei. For example, the binding protein can inhibit release of DNA from nuclei of neutrophils and/or neutrophil-like cells.

In another example, the binding protein is a di-scFv. In an example, the V_{H} and V_{L} of the di-scFv are separated by a linker comprising the sequence shown in SEQ ID NO: 17. In another example, the scFv's are separated by a linker. In this example, the linker may comprise a sequence shown in SEQ ID NO: 26 or 27. Accordingly, binding molecules of the disclosure can comprise a linker which comprises an amino acid sequence shown in any one of SEQ ID NO: 17; SEQ ID NO: 26 or SEQ ID NO: 27.

In an example, the binding protein is an intact antibody.

In an example, the inflammatory disease is selected from the group consisting of cystic fibrosis, ischemia, cardiovascular disease, periodontitis, fibrosis, pruritus, skin inflammation, psoriasis, multiple sclerosis, rheumatoid arthritis, osteoarthritis, systemic lupus erythematosus, Hashimoto's thyroidis, myasthenia gravis, diabetes type I or II, diabetic nephropathy, asthma, inflammatory liver injury, inflammatory glomerular injury, atopic dermatitis, allergic contact dermatitis, irritant contact dermatitis, seborrhoeic dermatitis, Sjoegren's syndrome, keratoconjunctivitis, uveitis, vasculitis, inflammatory bowel disease, Crohn's disease, ulcerative colitis, acute or chronic idiopathic inflammatory arthritis, myositis, a demyelinating disease, chronic obstructive pulmonary disease, interstitial lung disease, interstitial nephritis, chronic active hepatitis, gout, metabolic disease, inflammation associated with obesity, septic arthritis, aseptic arthritis, disseminated intravascular coagulation (DIC), Alzheimer's disease.

In another example, the inflammatory disease is selected from the group consisting of pneumonia, neutrophilic asthma, neutrophil-mediated anaphylaxis, inflammatory lung injury, chronic obstructive pulmonary disease (COPD).

In an example, the subject has sepsis secondary to their inflammatory disease. In an example, the subjects inflammation is caused by a bacterial infection. In an example, the inflammatory disorder is bacterial meningitis.

In another example, the inflammatory disease is an arthritis. In an example, the arthritis is rheumatoid arthritis. In an example, the arthritis is septic arthritis.

In an example, the subject has or is at risk of developing a thrombosis or an embolism. In another example, the thrombosis is a venous or arterial thrombosis. In another example, the embolism is a pulmonary embolism.

In another example, the inflammatory disease is Acute Respiratory Distress Syndrome (ARDS). In another example, the inflammatory disease is caused by a viral infection such as a rhinovirus, an influenza virus, a respiratory syncytial virus (RSV) or a coronavirus. In an example, the viral infection is caused by a coronavirus. In an example, the coronavirus is severe acute respiratory syndrome coronavirus (SARS-CoV), Middle East respiratory syndrome coronavirus (MERS-CoV) or severe acute respiratory syndrome coronavirus 2 (SARS-CoV-2). In a particular example, the inflammatory disease is coronavirus disease 19 (COVID-19).

In an example, the inflammatory disease is vasculitis. For example, the vasculitis may be ANCA associated vasculitis.

In an example, culture conditions of the disclosure comprise culturing neutrophil-like PLB-985 cells or neutrophils in culture medium which comprises an inflammatory stimulus. In an example, the inflammatory stimulus activates NOX-dependent NETosis (such as phorbol 12-myristate 13-acetate (PMA) or lipopolysaccharide (LPS)), or activatesNOX-independent NETosis (such as the calcium ionophore ionomycin (IM)). Accordingly, in an example, the inflammatory stimulus may be PMA or LPS. In another example, the inflammatory stimulus may be IM.

In an example, the inhibition of NET formation under culture conditions is determined based on one or both of:
1) level of cell death and extracellular DNA in NETs; and/or,
2) reduced DNA or NET release from neutrophil-like cells or neutrophils after culture with the inflammatory stimulus.

In another example, the present disclosure relates to a method of treating an inflammatory disease in a subject, the method comprising administering to the subject an effective amount of a cell penetrating, anti-DNA binding protein, wherein the cell penetrating, anti-DNA binding protein inhibits Neutrophil Extracellular Trap (NET) formation.

### BRIEF DESCRIPTION OF THE ACCOMPANYING DRAWINGS

FIG. 1. DX1 penetrates PLB-985 cells. Cells treated with control or 5 μM DX1 for one hour were washed, fixed, and immunostained to detect DX1. Representative images demonstrating penetration by DX1 into the cells are shown. Dark stain represents alkaline phosphatase-based detection of signal. Bar: 25 μm.

FIGS. 2A-2D. DAPI and SYTOX™ Green stains support the conclusion that DX1inhibits chromatin decondensation and DNA release by differentiated PLB-985 cells stimulated with PMA. (2A) Chromatin decondensation in differentiated PLB-985 cells after stimulation with PMA±a one hour pre-treatment with 10 μM DX1 was visualized by DAPI stain. Left panel: Control cells with decondensed DAPI stain after PMA stimulation. Right panel: DX1-treated cells retaining condensed DAPI signal after PMA stimulation. Bar: 25 μm. (2B) Quantification of the percentage of differentiated PLB-985 cells with decondensed DAPI stain after PMA stimulation. Control cells treated with PMA exhibited decondensed DAPI stain in 67.5±2.3% of cells after stimulation with PMA, compared to 37.8±3.8% of cells pre-treated with DX1 (N=2). (2C) DNA release by differentiated PLB-985 cells after stimulation with PMA±a one hour pre-treatment with 10 μM DX1 was visualized by addition of the impermeant DNA stain SYTOX™ Green. Representative images are shown. Bar: 25 μm. (2D) SYTOX™ Green signal in wells containing differentiated PLB-985 cells stimulated with PMA±a one hour pre-treatment with 10 μDX1 was quantified by plate reader. Pre-treatment with DX1reduced SYTOX™ Green signal to 0.40±0.01 relative to control (****P<0.0001, N=3), consistent with a DX1-mediated inhibition of DNA release.

FIGS. 3A-3B. Differentiated PLB-985 respond to PMA and PAD4 inhibitor as expected. (3A) Differentiated PLB-985 cells release DNA when stimulated by PMA, consistent with expected NETosis response. Concentration of DNA adherent to wells after treatment of undifferentiated and neutrophil-like PLB-985 cells with control or PMA was determined by spectrophotometry. Differentiated, but not undifferentiated, PLB-985 cells yielded significantly greater amounts of released/adherent DNA after stimulation with PMA. (N=3) (3B) GSK484 inhibits DNA release by differentiated PLB-985 cells after stimulation with PMA. Differentiated PLB-985 cells were treated with control or the PAD4 inhibitor GSK484 (G1, G10, G150: 1, 10, or 50 μM GSK484) for one hour prior to stimulation with PMA, followed by measurement of DNA adherent to wells by spectrophotometry. Cells treated with GSK484 showed a dose-dependent inhibition of DNA release in response to PMA, as expected. (N=2).

FIG. 4. DX1 inhibits release of DNA from differentiated PLB-985 cells stimulated with PMA. Differentiated PLB-985 cells treated with control buffer or DX1 were left unstimulated or stimulated by addition of PMA, followed by measurement of adherent DNA by spectrophotometry. PMA stimulation of control cells increased the amount of released/adherent DNA to 4.50±0.14 (****P<0.0001, N=3) relative to unstimulated control cells. Response to PMA was inhibited in cells treated with DX1, with DNA content 1.80±0.11 relative to unstimulated control cells (****P<0.0001, N=3).

FIGS. 5A-5B. DX1 does not inhibit citrullination of histone H3 in differentiated PLB-985 cells. Differentiated PLB-985 cells were pre-treated with control media or media containing 10 μM DX1 for one hour, followed by stimulation with PMA. Cellular contents were isolated and analyzed by H3Cit western blot. Representative cropped blot is shown in (5A), and quantification of H3Cit band intensity normalized to actin in (5B). Addition of PMA appeared to reduce H3Cit content to 0.64±0.06 (N=2) relative to unstimulated cells, likely reflecting the H3Cit release in NETosis. Pre-treatment with DX1 did not yielded any apparent reduction in citrullination of H3, but rather may have caused an increase in intracellular H3Cit content to 1.46±0.20 relative to unstimulated cells (N=3). These data are consistent with inhibition of NET release by DX1.

FIGS. 6A and 6B. DX1 does not stimulate NETosis in mouse neutrophils. (6A) Mouse neutrophils were treated with control media or media containing 10 μM DX1 for 30 minutes, and NET formation was visualized by DAPI and immunostaining for NET markers MPO, H3Cit, and PAD4. Representative images are shown. (6B) The percentage of NETs visualized in control and DX1-treated cells was 3.5%±1.0 and 4.0% 35 1.5 (P=0.79, N=4), respectively. These results demonstrate that DX1 does not stimulate NETosis in mouse neutrophils.

FIGS. 7A and 7B. DX1 inhibits NOX-dependent NETosis in mouse neutrophils stimulated with PMA. (7A) Mouse neutrophils were treated with control media or media containing 10 μM DX1 for 30 minutes, followed by stimulation of NOX-dependent NETosis by addition of PMA. NET formation was visualized by immunostaining for NET markers MPO, H3Cit, and PAD4. Representative images are shown. (7B) The percentage of NETs visualized in control and DX1-treated cells was 36.3%±11.1 and 8.3%±2.7 (*P=0.05, N=4). These results demonstrate that DX1 inhibits NOX-dependent NETosis in mouse neutrophils treated with PMA.

FIGS. 8A and 8B. DX1 inhibits NOX-independent NETosis in mouse neutrophils stimulated with the calcium ionophore IM. (8A) Mouse neutrophils were treated with control media or media containing 10 μM DX1 for 30 minutes, followed by stimulation of NOX-independent NETosis by addition of the calcium ionophore IM. NET formation was visualized by immunostaining for NET markers MPO, H3Cit, and PAD4. Representative images are shown. (8B) The percentage of NETs visualized in control and DX1-treated cells was 15.5%±3.1 and 2.3%±0.8 (*P<0.01, N=4). These results demonstrate that DX1 inhibits NOX-independent NETosis in mouse neutrophils treated with IM.

FIG. 9. Graphic depiction of an experiment testing the impact of DX1 in a 20-day mouse model of ANCA vasculitis.

FIGS. 10A-10D. DX1 significantly reduces kidney damage and immune infiltration in a 20-day mouse model of ANCA vasculitis. Mice were treated as depicted in FIG. 9. Glomerular segmental necrosis, macrophages, neutrophils, and CD4+ T-cells was evaluated in control and MPO-immunized mice that subsequently were treated with or without one or two doses of DX1. Mice immunized with MPO and treated with DX1 had significantly reduced amounts of all of the signs of glomerular inflammation as compared to mice immunized with MPO and treated with control. These findings demonstrate that DX1 suppresses damage associated with an inflammatory disease, in this case ANCA vasculitis, in vivo.

### KEY TO SEQUENCE LISTING

SEQ ID NO: 1—Heavy chain CDR1 of murine 3E10

SEQ ID NO: 2—Heavy chain CDR2 of murine 3E10

SEQ ID NO: 3—Heavy chain CDR3 of murine 3E10

SEQ ID NO: 4—Light chain CDR1 of murine 3E10

SEQ ID NO: 5—Light chain CDR2 of murine 3E10

SEQ ID NO: 6—Light chain CDR3 of murine 3E10

SEQ ID NO: 7—V_{H} chain of murine 3E10

SEQ ID NO: 8—V_{L} chain of murine 3E10

SEQ ID NO: 9—Heavy chain CDR1 of humanized antibody DX1/DX3, VH_1 and VH_2

SEQ ID NO: 10—Heavy chain CDR1 of humanized antibody VH_3m

SEQ ID NO: 11—Heavy chain CDR2 of humanized antibody DX1/DX3, VH_1 and VH_2

SEQ ID NO: 12—Heavy chain CDR2 of humanized antibody VH_3m

SEQ ID NO: 13—Heavy chain CDR3 of humanized antibody DX1/DX3, VH_1, VH_2 and VH_3m

SEQ ID NO: 14—Light chain CDR1 of humanized antibody DX1/DX3, VH_1 and VH_2

SEQ ID NO: 15—Light chain CDR1 of humanized antibody VH_3m

SEQ ID NO: 16—Light chain CDR2 of humanized antibody DX1/DX3, VH_1, VH_2 and VH_3m

SEQ ID NO: 17—Light chain CDR3 of humanized antibody DX1/DX3 VH_1, VH_2 and VH_3m

SEQ ID NO: 18—V_{H} chain of humanized antibody (DX1/DX3)

SEQ ID NO: 19—V_{H} chain of humanized antibody VH_1

SEQ ID NO: 20—V_{H} chain of humanized antibody VH_2

SEQ ID NO: 21—V_{H} chain of humanized antibody VH_3m

SEQ ID NO: 22—V_{L} chain of humanized antibody (DX1/DX3)

SEQ ID NO: 23—V_{L} chain of humanized antibody VH_1

SEQ ID NO: 24—V_{L} chain of humanized antibody VH_2

SEQ ID NO: 25—V_{L} chain of humanized antibody VH_3m

SEQ ID NO: 26—Linker sequence 1

SEQ ID NO: 27—Linker sequence 2

SEQ ID NO: 28—Hinge sequence

SEQ ID NO: 29—Signal sequence

SEQ ID NO: 30—V_{H} chain of 5C6

SEQ ID NO: 31—Heavy chain CDR1 of 5C6

SEQ ID NO: 32—Heavy chain CDR2 of 5C6

SEQ ID NO: 33—Heavy chain CDR3 of 5C6

SEQ ID NO: 34—V_{L} chain of 5C6

SEQ ID NO: 35—Light chain CDR1 of 5C6

SEQ ID NO: 36—Light chain CDR2 of 5C6

SEQ ID NO: 37—Light chain CDR3 of 5C6

SEQ ID NO: 38—DX1 sequence

SEQ ID NO: 39—DX3 sequence

SEQ ID NO: 40—Alternate heavy chain CDR1 of 3E10

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art (e.g., molecular biology, biochemistry, antibodies, antibody fragments such as single chain fragment variable and clinical studies).

The term “cell penetrating” is used in the context of the present disclosure to refer to an anti-DNA binding protein that is transported into the cytoplasm of living mammalian cells and, preferably, binds DNA (e.g., single-stranded and/or double-stranded DNA). In an example, cell penetrating binding proteins also penetrate cell nuclei. Accordingly, binding proteins of the disclosure can localise in cell nuclei and bind DNA (i.e. they are nuclear penetrating). Such binding proteins are distinguished from antibodies that can penetrate into cells but remain sequestered in the cytoplasm.

The term “anti-DNA binding protein” is used in the context of the present disclosure to refer to antibodies capable of binding DNA.

The term “binding protein” is used in the context of the present disclosure to refer to human or humanised immunoglobulin molecules immunologically reactive with a particular antigen and includes both polyclonal and monoclonal antibodies. The term “binding protein” also includes antigen binding forms of antibodies, including fragments with antigen-binding capability (e.g., Fab′, F(ab′)₂, Fab, Fv and rIgG as discussed in Pierce Catalogue and Handbook, 1994-1995 (Pierce Chemical Co., Rockford, Ill.); Kuby, J., Immunology, 3^{rd} Ed., W. H. Freeman & Co., New York (1998). The term is also used to refer to recombinant single chain Fv fragments (scFv) as well as divalent (di-scFv) and trivalent (tri-scFV) forms thereof. The term antibody also includes diabodies, triabodies, and tetrabodies.

The term binding protein as used herein encompasses binding proteins which comprise an antibody such as a bi-specific molecule. For example, a binding protein may comprise an above referenced immunoglobulin such as an antibody and an above referenced fragment such as an Fv.

An “antigen binding fragment” of an antibody comprises one or more variable regions of an intact antibody. Examples of antibody fragments include Fab, Fab′, F(ab′)2 and Fv fragments; diabodies; linear antibodies and single-chain antibody molecules formed from antibody fragments. For example, the term antigen binding fragment may be used to refer to recombinant single chain Fv fragments (scFv) as well as divalent (di-scFv) and trivalent (tri-scFV) forms thereof. In an example, the binding protein is an antigen binding fragment. Such fragments can be produced via various methods known in the art.

The term “immunoglobulin” will be understood to include binding proteins of the disclosure, such as anti-DNA binding proteins, which comprise an immunoglobulin domain. Exemplary immunoglobulins are antibodies. Additional proteins encompassed by the term “immunoglobulin” include domain antibodies, camelid antibodies and antibodies from cartilaginous fish (i.e., immunoglobulin new antigen receptors (IgNARs)). Generally, camelid antibodies and IgNARs comprise a V_{H}, however lack a V_{L} and are often referred to as heavy chain immunoglobulins.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains. In an example, whole antibodies include an Fc region. The constant domains may be wild-type sequence constant domains (e.g., human wild-type sequence constant domains) or amino acid sequence variants thereof. In an example, the antibody is an IgG.

As used herein, “variable region” refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region typically has three CDR regions identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system” or “Kabat”.

Other conventions that include corrections or alternate numbering systems for variable domains include IMGT (Lefranc, et al. (2003), Dev Comp Immunol 27: 55-77), Chothia (Chothia C, Lesk AM (1987), J Mal Biol 196: 901-917; Chothia, et al. (1989), Nature 342: 877-883) and AHo (Honegger A, Plückthun A (2001) J Mol Biol 309: 657-670). For convenience, examples of antibodies of the present disclosure may also be labeled according to IMGT.

“Framework regions” (Syn. FR) are those variable domain residues other than the CDR residues.

The term “constant region” as used herein, refers to a portion of heavy chain or light chain of an antibody other than the variable region. In a heavy chain, the constant region generally comprises a plurality of constant domains and a hinge region, e.g., a IgG constant region comprises the following linked components, a constant heavy C_{H}1, a linker, a C_{H}2 and a C_{H}3. In a heavy chain, a constant region comprises a Fc. In a light chain, a constant region generally comprise one constant domain (a CL1).

The term “fragment crystalizable” or “Fc” or “Fc region” or “Fc portion” (which can be used interchangeably herein) refers to a region of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Exemplary heavy chain constant regions are gamma 1 (IgG1), gamma 2 (IgG2) and gamma 3 (IgG3), or hybrids thereof.

A “constant domain” is a domain in an antibody the sequence of which is highly similar in antibodies/antibodies of the same type, e.g., IgG or IgM or IgE. A constant region of an antibody generally comprises a plurality of constant domains, e.g., the constant region of γ, α or δ heavy chain comprises two constant domains.

The term “conjugated” is used in the context of the present disclosure to refer to binding proteins of the present disclosure that are conjugated to another compound, e.g., therapeutic compound or a diagnostic compound. Accordingly, in one example, the binding protein of the present disclosure are “conjugated”. The nature of the conjugation is not particularly limited so long as it maintains the capacity of the binding protein to inhibit Neutrophil Extracellular Trap (NET) formation under culture conditions.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill of those practicing in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, the term “binds” in reference to the interaction of a binding protein and an antigen means that the interaction is dependent upon the presence of a particular structure (e.g., an antigenic determinant or epitope) on the antigen. For example, a binding protein recognizes and binds to a specific antigen structure rather than to antigens generally. For example, if a binding protein binds to epitope “A”, the presence of a molecule containing epitope “A” (or free, unlabeled “A”), in a reaction containing labeled “A” and the binding protein, will reduce the amount of labeled “A” bound to the binding protein.

As used herein, the term “specifically binds” shall be taken to mean that the binding interaction between the binding protein and DNA is dependent on detection of the DNA by the binding protein. Accordingly, the binding protein preferentially binds or recognizes DNA even when present in a mixture of other molecules or organisms.

In one example, the binding protein reacts or associates more frequently, more rapidly, with greater duration and/or with greater affinity with DNA than it does with alternative antigens or cells. It is also understood by reading this definition that, for example, the binding protein that specifically binds to DNA may or may not specifically bind to a second antigen. As such, “specific binding” does not necessarily require exclusive binding or non-detectable binding of another antigen. The term “specifically binds” can be used interchangeably with “selectively binds” herein. Generally, reference herein to binding means specific binding, and each term shall be understood to provide explicit support for the other term. Methods for determining specific binding will be apparent to the skilled person. For example, a binding protein of the disclosure is contacted with DNA or an alternative antigen. Binding of the binding protein to DNA or alternative antigen is then determined and the binding protein that binds as set out above to the DNA rather than the alternative antigen is considered to specifically bind to DNA.

Binding proteins according to the present disclosure and compositions comprising the same can be administered to a subject to treat various indications such as inflammatory diseases. Terms such as “subject”, “patient” or “individual” are terms that can, in context, be used interchangeably in the present disclosure. In an example, the subject is a mammal. The mammal may be a companion animal such as a dog or cat, or a livestock animal such as a horse or cow. In one example, the subject is a human. For example, the subject can be an adult. In another example, the subject can be a child. In another example, the subject can be an adolescent. In one example, the subject has an inflammatory disease. For example, the subject may have a genetic disease such as cystic fibrosis or a neurological disorder with an underlying inflammatory component such as multiple sclerosis.

In an example, the subject does not have cancer. In an example, the subject does not have lupus.

In an example, the subject has or is at risk of developing a thrombosis or an embolism. In this example, methods of the present disclosure can encompass treatment or prophylaxis for a thrombosis or an embolism.

The term “thrombosis” is used herein to refer to the formation of a thrombus or blood clot. In an example, the thrombosis is “arterial thrombosis” where the blood clot develops in an artery. Such blood clots are particularly dangerous to a subject as they can obstruct blood flow to major organs such as the heart or brain. In an example, the thrombosis is “venous thrombosis” where the blood clot develops in a vein.

The term “pulmonary embolism” is used herein to refer to a blockage of an artery in the lungs by a substance that has moved from elsewhere in the body through the bloodstream.

Subjects treated according to the present disclosure may have symptoms indicative of inflammatory disease. Exemplary symptoms may include fatigue, trouble breathing, shortness of breath, inability or decreased ability to exercise, coughing with or without blood or mucus, pain when breathing in or out, wheezing, chest tightness, unexplained weight loss, and musculoskeletal pain.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disease are mitigated or eliminated. In an example, treatment is characterised by a reduction of inflammatory markers in a subject. In addition, the term “treatment” includes palliative treatment, that is, treatment designed for the relief of symptoms rather than the curing of the disease, pathological condition, or disorder; prophylactic treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevention” includes providing prophylaxis with respect to occurrence or recurrence of a disease in an individual. An individual may be predisposed to or at risk of developing the disease or disease relapse but has not yet been diagnosed with the disease or the relapse.

In an example, methods of the present disclosure inhibit disease progression or disease complication in a subject. In an example, methods of the disclosure inhibit progression or severity in an inflammatory disease. In an example, the methods of the present disclosure inhibit NET formation in a subject. In this example, NET formation can be reduced relative to an untreated subject or the level of NET formation in the subject prior to administering a binding protein disclosed herein.

An “effective amount” refers to at least an amount effective, at dosages and for periods of time necessary, to achieve the desired therapeutic, prophylactic, diagnostic or otherwise informative result. An effective amount can be provided in one or more administrations. In some examples of the present disclosure, the term “effective amount” is meant an amount necessary to effect treatment of a disease or condition described below. The effective amount may vary according to the disease or condition to be treated and also according to the weight, age, racial background, sex, health and/or physical condition and other factors relevant to the subject being treated. Typically, the effective amount will fall within a relatively broad range (e.g. a “dosage” range) that can be determined through routine trial and experimentation by a medical practitioner. The effective amount can be administered in a single dose or in a dose repeated once or several times over a treatment period. It is understood that the specific dose level for any particular patient depends upon a variety of factors including the activity of the specific antibody employed, the age, body weight, general health, sex, diet, time of administration, route of administration, and rate of excretion, drug combination and the severity of the particular disease undergoing therapy.

A “therapeutically effective amount” is at least the minimum concentration required to effect a measurable improvement of a particular disorder (e.g. inflammatory disease). A therapeutically effective amount herein may also vary according to factors such as the disease state, age, sex, and weight of the patient, and the ability of the antibody to elicit a desired response in the individual. A therapeutically effective amount is also one in which any toxic or detrimental effects of the binding protein are outweighed by the therapeutically beneficial effects. In the case of inflammatory disease, the therapeutically effective amount of the binding protein may inhibit (i.e., slow to some extent and, in some examples, stop) disease symptoms, disease progression; and/or relieve to some extent one or more of the symptoms associated with the inflammatory disease being treated. For inflammatory disease, efficacy in vivo can, for example, be measured by assessing the duration of survival, time to disease progression (TTP), the response rates (RR), duration of response, and/or quality of life.

Binding proteins of the disclosure inhibit Neutrophil Extracellular Trap (NET) formation. In an example, capability of binding proteins disclosed herein to inhibit Neutrophil Extracellular Trap (NET) formation is determined under culture conditions. The term “culture conditions” is used to refer to cells growing in culture. In an example, culture conditions refers to a population of cells in cell culture such as neutrophils or neutrophil-like cells such as PLB-985, HL-60 cells (e.g. ATCC CCL-240 or a sub clone thereof), NB4. In an example, culture conditions refers to a culture expanded population of cells. In an example, culture conditions refers to an actively dividing population of cells. Such cells may, in an example, be in exponential growth phase. In an example, culture conditions comprises culturing cells with a stimulus of NET formation such as phorbol 12-myristate 13-acetate (PMA) or lipopolysaccharide (LPS). Other exemplary stimulus of NET formation include calcium ionophores such as A23187 (A23) or inomycin. In an example, the stimulus promotes NOX-dependent NET formation. In another example, the stimulus promotes NOX-independent NET formation. In an example, culture conditions comprises culturing cells with 300 nM PMA for 4.5 hours. In another example, culture conditions comprise culturing cells with calcium ionophore (ionomycin, IM; e.g. 4 μM).

NET formation can also be measured under culture conditions. In an example, the level of a particular marker that is representative of NET formation can be determined by taking a sample of cell culture media and measuring the level of marker in the sample. In another example, the level of a particular marker can be determined by taking a sample of cells and measuring the level of the marker in the cell lysate. Those of skill in the art will appreciate that secreted markers can be measured by sampling the culture media while markers expressed on the surface or inside cultured cell(s) may be measured by assessing a sample of cell lysate. In an example, the sample is taken when the cells are in exponential growth phase. In an example, the sample is taken after at least two days in culture. In another example, the level of a particular marker can be determined by visually assessing cells under culture conditions. In an example, the marker of NET formation is level of cell death and extracellular DNA in NETs. In another example, the marker of NET formation is DNA release from neutrophil-like cells after culture with the inflammatory stimulus. In this example, inhibition of NET formation is characterised by reduced DNA release from neutrophil-like cells after culture with the inflammatory stimulus. Various markers of Net formation are known in the art. Examples include circulating cell free DNA, myeloperoxidase (MPO), Citrullinated histone 3 (H3Cit), and neutrophil elastase (NE). In an example, the capability of the cell penetrating, anti-DNA binding protein to inhibit both NOX-dependent and/or NOX-independent NET formation can be assessed following neutrophil stimulation with either phorbol 12-myristate 13-acetate (PMA) or ionomycin (IM).

In an example, binding proteins of the disclosure inhibit release of Citrullinated histone H3 (H3Cit) from neutrophil-like cells under culture conditions. Various methods are available for determining the level of H3Cit under culture conditions such as Western Blot. In an example, binding proteins of the disclosure change serum levels of H3Cit in a subject after administration. In this example, a binding protein of the disclosure is administered to a subject, a serum sample is subsequently obtained and the level H3Cit in the sample is compared to a baseline level of H3Cit from the subject to determine whether H3Cit levels have been changed.

### Cell Penetrating Anti-DNA Binding Proteins

The present disclosure relates to anti-DNA binding proteins that inhibit Neutrophil Extracellular Trap (NET) formation under culture conditions. The term “Neutrophil Extracellular Trap (NET)” is used in the context of the present disclosure to refer to a composition comprising extracellular chromatin decorated with histones and numerous granular proteins. In an example, NET components are intermixed amongst a scaffold of nuclear and/or mitochondrial DNA. “NET formation” and “NETosis” are used in the context of the present disclosure to describe the sequence of cellular events leading up to the active release of NETs. In an example, the process is characterised by regulated neutrophil cell death.

In an example, anti-DNA binding protein is also cell penetrating. Accordingly, the present disclosure contemplates use of cell penetrating, anti-DNA binding proteins. In an example, the cell penetrating, anti-DNA binding protein is also nuclear penetrating. In other words, the binding protein can enter the nucleus of a cell and bind DNA rather than remaining sequestered in the cytoplasm of a cell. In this example, the anti-DNA binding protein can be referred to as a nuclear penetrating anti-DNA binding protein. In an example, the nuclear penetrating anti-DNA binding protein inhibits release of DNA from cell nuclei. For example, the nuclear penetrating anti-DNA binding protein can inhibit release of DNA from nuclei of neutrophil and/or neutrophil like cells. In an example, inhibition of DNA release from cell nuclei is determined under culture conditions.

In one example, the binding protein is an autoantibody derived from a subject or an animal with an autoimmune disease. In an example, the autoantibody is derived from a subject with systemic lupus erythematous, or an animal model thereof. The term “derived” as used herein encompasses recombinant forms of an antibody of the disclosure produced using recombinant techniques such as the methods discussed below. For example, a nucleic acid sequence encoding an autoantibody from a subject with systemic lupus erythematous, or an animal model thereof can be provided in a recombinant system to produce a recombinant form of the antibody. In an example, the autoantibody binds DNA. In an example, the autoantibody is cell penetrating. Examples of anti-DNA autoantibodies are known in the art (Hansen et al. (2012) Sci Transl Med., 4:157ral42; Noble et al. (2015) Cancer Research., 75:2285-2291; Noble et al. (2016) Nat Rev Rheumatol., 12:429-34). In an example, the autoantibody is 3E10 (i.e. antibody having a VH comprising SEQ ID NO: 7 and a VL comprising SEQ ID NO: 8) or a humanised form thereof.

In one example, an anti-DNA binding protein according to the present disclosure comprises a heavy chain variable region (V_{H}) having a CDR 1 as shown in SEQ ID NO: 1, a CDR2 as shown in SEQ ID NO: 2, a CDR3 as shown in SEQ ID NO: 3 and a light chain variable region (V_{L}) having a CDR1 as shown in SEQ ID NO: 4, a CDR2 as shown in SEQ ID NO: 5 and a CDR3 as shown in SEQ ID NO: 6 or a humanized form thereof. Accordingly, in an example, the present disclosure encompasses an binding protein which comprises a heavy chain variable region (V_{H}) having a CDR 1 as shown in SEQ ID NO: 1, a CDR2 as shown in SEQ ID NO: 2, a CDR3 as shown in SEQ ID NO: 3 and a light chain variable region (V_{L}) having a CDR1 as shown in SEQ ID NO: 4, a CDR2 as shown in SEQ ID NO: 5 and a CDR3 as shown in SEQ ID NO: 6 or a humanized form thereof. In an example, the binding protein competes for binding to DNA with an binding protein which comprises a heavy chain variable region (V_{H}) having a CDR 1 as shown in SEQ ID NO: 1, a CDR2 as shown in SEQ ID NO: 2, a CDR3 as shown in SEQ ID NO: 3 and a light chain variable region (V_{L)} having a CDR1 as shown in SEQ ID NO: 4, a CDR2 as shown in SEQ ID NO: 5 and a CDR3 as shown in SEQ ID NO: 6 or a humanized form thereof. In these examples, the V_{H} CDR1 may rather comprise SEQ ID NO: 40.

In an example, the binding protein inhibits DNA repair. Inhibition of DNA repair can be assessed in-vitro by contacting cells with a DNA damaging agent and the binding protein before measuring the capacity of cells to repair DNA. In an example, the cells are cancer cells. In an example, inhibition of DNA repair is assessed based on the level of apoptosis in damaged cells.

Binding proteins of the disclosure such as antibodies or fragments thereof are particularly useful in that they can inhibit NET formation. Accordingly, in an example, the present disclosure encompasses an antibody or fragment thereof which comprises a heavy chain variable region (V_{H}) having a CDR 1 as shown in SEQ ID NO: 1, a CDR2 as shown in SEQ ID NO: 2, a CDR3 as shown in SEQ ID NO: 3 and a light chain variable region (V_{L}) having a CDR1 as shown in SEQ ID NO: 4, a CDR2 as shown in SEQ ID NO: 5 and a CDR3 as shown in SEQ ID NO: 6 or a humanized form thereof, wherein, the antibody or fragment thereof inhibits Neutrophil Extracellular Trap (NET) formation under culture conditions. In an example, the V_{H} CDR1 may rather comprise SEQ ID NO: 40.

The present disclosure also encompasses humanized forms and CDR variants of the above referenced example. Accordingly in another example, the humanized form of the antibody or fragment thereof comprises a V_{H} having a CDR1 as shown in SEQ ID NO: 9 or SEQ ID NO: 10, a CDR2 as shown in SEQ ID NO: 11 or SEQ ID NO: 12, a CDR3 as shown in SEQ ID NO: 13 and a V_{L} having a CDR1 as shown in SEQ ID NO: 14 or SEQ ID NO: 15, a CDR2 as shown in SEQ ID NO: 16 and a CDR3 as shown in SEQ ID NO: 17 or a variant thereof that competes for binding to DNA with an antibody which comprises a V_{H} having a CDR1 as shown in SEQ ID NO: 9 or SEQ ID NO: 10, a CDR2 as shown in SEQ ID NO: 11 or SEQ ID NO: 12, a CDR3 as shown in SEQ ID NO: 13 and a V_{L} having a CDR1 as shown in SEQ ID NO: 14 or SEQ ID NO: 15, a CDR2 as shown in SEQ ID NO: 16 and a CDR3 as shown in SEQ ID NO: 17.

In another example, the humanized form of the antibody or fragment thereof comprises a V_{H} having a CDR1 as shown in SEQ ID NO: 9, a CDR2 as shown in SEQ ID NO: 11, a CDR3 as shown in SEQ ID NO: 13 and a V_{L} having a CDR1 as shown in SEQ ID NO: 14, a CDR2 as shown in SEQ ID NO: 16 and a CDR3 as shown in SEQ ID NO: 17 or a variant thereof that competes for binding to DNA with an antibody which comprises a V_{H} having a CDR1 as shown in SEQ ID NO: 9, a CDR2 as shown in SEQ ID NO: 11, a CDR3 as shown in SEQ ID NO: 13 and a V_{L} having a CDR1 as shown in SEQ ID NO: 14, a CDR2 as shown in SEQ ID NO: 16 and a CDR3 as shown in SEQ ID NO: 17. In another example, the humanized form of the antibody or fragment thereof comprises a V_{H} having a CDR1 as shown in SEQ ID NO: 9, a CDR2 as shown in SEQ ID NO: 11, a CDR3 as shown in SEQ ID NO: 13 and a V_{L} having a CDR1 as shown in SEQ ID NO: 14, a CDR2 as shown in SEQ ID NO: 16 and a CDR3 as shown in SEQ ID NO: 17.

In another example, the humanized form of the antibody or fragment thereof comprises a V_{H} having a CDR1 as shown in SEQ ID NO: 10, a CDR2 as shown in SEQ ID NO: 12, a CDR3 as shown in SEQ ID NO: 13 and a V_{L} having a CDR1 as shown in SEQ ID NO: 15, a CDR2 as shown in SEQ ID NO: 16 and a CDR3 as shown in SEQ ID NO: 17 or a variant thereof that competes for binding to DNA with an antibody which comprises a V_{H} having a CDR1 as shown in SEQ ID NO: 10, a CDR2 as shown in SEQ ID NO: 12, a CDR3 as shown in SEQ ID NO: 13 and a V_{L} having a CDR1 as shown in SEQ ID NO: 15, a CDR2 as shown in SEQ ID NO: 16 and a CDR3 as shown in SEQ ID NO: 17. In the above referenced examples, the CDRs are subject to at least one amino acid substitution. In another example, the CDRs are subject to at least two amino acid substitutions. In another example, the CDRs are subject to at least three amino acid substitutions. In an example, the substitution(s) are in CDR1. In another example, the substitution(s) are in VH CDR1. In another example, the substitution(s) are in VL CDR2. In another example, the substitution(s) are in VH CDR2.

In another example, the antibody or fragment thereof comprises a V_{H} having a CDR1 as shown in SEQ ID NO: 6, a CDR2 as shown in SEQ ID NO: 7 and a CDR3 as shown in SEQ ID NO: 8 or a humanized form thereof.

In one example, the humanized form of the antibody or fragment thereof comprises a V_{H} which comprises an amino acid sequence as shown in any one of SEQ ID NOs: 18-21 and a V_{L} as shown in any one of SEQ ID NOs: 22-25 or a variant thereof that competes for binding to DNA with an antibody which comprises a V_{H} which comprises an amino acid sequence as shown in any one of SEQ ID NOs: 18-21 and a V_{L} as shown in any one of SEQ ID NOs: 22-25.

In one example, the humanized form of the antibody or fragment thereof comprises a V_{H} which comprises an amino acid sequence as shown in SEQ ID NO: 18 and a V_{L} as shown in SEQ ID NO: 22 or a variant thereof that competes for binding to DNA with an antibody which comprises a V_{H} which comprises an amino acid sequence as shown in SEQ ID NO: 18 and a V_{L} as shown in SEQ ID NO: 22. In an example, the humanized form of the antibody or fragment thereof comprises a V_{H} which comprises an amino acid sequence as shown in SEQ ID NO: 18 and a V_{L} as shown in SEQ ID NO: 22.

In one example, the humanized form of the antibody or fragment thereof comprises a V_{H} which comprises an amino acid sequence as shown in SEQ ID NO: 19 and a V_{L} as shown in SEQ ID NO: 23 or a variant thereof that competes for binding to DNA with an antibody which comprises a V_{H} which comprises an amino acid sequence as shown in SEQ ID NO: 19 and a V_{L} as shown in SEQ ID NO: 23.

In one example, the humanized form of the antibody or fragment thereof comprises a V_{H} which comprises an amino acid sequence as shown in SEQ ID NO: 20 and a V_{L} as shown in SEQ ID NO: 24 or a variant thereof that competes for binding to DNA with an antibody which comprises a V_{H} which comprises an amino acid sequence as shown in SEQ ID NO: 20 and a V_{L} as shown in SEQ ID NO: 24.

In one example, the humanized form of the antibody or fragment thereof comprises a V_{H} which comprises an amino acid sequence as shown in SEQ ID NO: 21 and a V_{L} as shown in SEQ ID NO: 25 or a variant thereof that competes for binding to DNA with an antibody which comprises a V_{H} which comprises an amino acid sequence as shown in SEQ ID NO: 21 and a V_{L} as shown in SEQ ID NO: 25.

Other examples of suitable anti-DNA binding proteins are known in the art (Zack et al. (1995) J. Immunol., 154:1987-1994; Gu et al. (1998) J. Immunol., 161:6999-7006; Noble et al. (2014) Sci Rep., 4:5958; ATCC Accession No. PTA 2439 hybridoma; WO2019/018426). In an example, the antibody is not an anti-guanosine antibody.

In an example, the anti-DNA binding protein can be a fragment such as a cell penetrating anti-DNA Fv. In an example, the Fv is a scFv. In an example, the fragment has an antigen binding domain, wherein the antigen binding domain binds to or specifically binds to DNA. For example, the Fv can bind the same epitope as an antibody having a V_{H} comprising an amino acid sequence as shown in SEQ ID NO: 7 and a V_{L} comprising an amino acid sequence as shown in SEQ ID NO: 8. In another example, the Fv can bind the same epitope as an antibody having a V_{H} comprising an amino acid sequence as shown in SEQ ID NO: 18 and a V_{L} comprising an amino acid sequence as shown in SEQ ID NO: 22. In another example, the Fv can bind the same epitope as an antibody having a V_{H} comprising an amino acid sequence as shown in SEQ ID NO: 19 and a V_{L} comprising an amino acid sequence as shown in SEQ ID NO: 23. In another example, the Fv can bind the same epitope as an antibody having a V_{H} comprising an amino acid sequence as shown in SEQ ID NO: 20 and a V_{L} comprising an amino acid sequence as shown in SEQ ID NO: 24. In another example, the Fv can bind the same epitope as an antibody having a V_{H} comprising an amino acid sequence as shown in SEQ ID NO: 21 and a V_{L} comprising an amino acid sequence as shown in SEQ ID NO: 25. In an example, the Fv is a scFv.

In an example, the Fv comprises a linker. Various suitable linkers and methods for their design have been described previously (e.g. U.S. Pat. No. 4,946,778; WO 1994/012520; and U.S. Pat. No. 4,704,692). In an example, the Fv comprises a glycine-serine (GS) linker. In one example, the Fv comprises a linker comprising the sequence as shown in SEQ ID NO: 26.

In an example, the V_{H} and V_{L} of the Fv can be in a single polypeptide chain. In another example, the Fv lacks an Fc region. For example, the Fv can be a single chain Fv fragment (scFv), a dimeric scFv (di-scFv), a trimeric scFv (tri-scFv). In an example, the Fv is an scFv. In another example, the Fv is a di-scFv. The scFvs may be separated by a linker. In one example, the linker comprises the sequence shown in SEQ ID NO: 27.

Thus, in an example, the binding protein may be a scFv comprising a V_{H} which comprises an amino acid sequence as shown in SEQ ID NO: 18 and a V_{L} as shown in SEQ ID NO: 22 or a variant thereof that competes for binding to DNA with an antibody which comprises a V_{H} which comprises an amino acid sequence as shown in SEQ ID NO: 18 and a V_{L} as shown in SEQ ID NO: 22 and a linker separating the scFvs comprising the sequence shown in SEQ ID NO: 27.

In another example, the binding protein may be a scFv comprising a V_{H} which comprises an amino acid sequence as shown in SEQ ID NO: 19 and a V_{L} as shown in SEQ ID NO: 23 or a variant thereof that competes for binding to DNA with an antibody which comprises a V_{H} which comprises an amino acid sequence as shown in SEQ ID NO: 19 and a V_{L} as shown in SEQ ID NO: 23 and a linker separating the scFvs comprising the sequence shown in SEQ ID NO: 27.

In another example, the binding protein may be a scFv comprising a V_{H} which comprises an amino acid sequence as shown in SEQ ID NO: 20 and a V_{L} as shown in SEQ ID NO: 24 or a variant thereof that competes for binding to DNA with an antibody which comprises a V_{H} which comprises an amino acid sequence as shown in SEQ ID NO: 20 and a V_{L} as shown in SEQ ID NO: 24 and a linker separating the scFvs comprising the sequence shown in SEQ ID NO: 27.

In another example, the binding protein may be a scFv comprising a V_{H} which comprises an amino acid sequence as shown in SEQ ID NO: 21 and a V_{L} as shown in SEQ ID NO: 25 or a variant thereof that competes for binding to DNA with an antibody which comprises a V_{H} which comprises an amino acid sequence as shown in SEQ ID NO: 21 and a V_{L} as shown in SEQ ID NO: 25 and a linker separating the scFvs comprising the sequence shown in SEQ ID NO: 27.

Thus, in another example, the binding protein may be a di-scFV comprising a V_{H} which comprises an amino acid sequence as shown in SEQ ID NO: 18 and a V_{L} as shown in SEQ ID NO: 22 or a variant thereof that competes for binding to DNA with an antibody which comprises a V_{H} which comprises an amino acid sequence as shown in SEQ ID NO: 18 and a V_{L} as shown in SEQ ID NO: 22 and a linker separating the scFvs comprising the sequence shown in SEQ ID NO: 27.

In another example, the binding protein may be a di-scFv comprising a V_{H} which comprises an amino acid sequence as shown in SEQ ID NO: 19 and a V_{L} as shown in SEQ ID NO: 23 or a variant thereof that competes for binding to DNA with an antibody which comprises a V_{H} which comprises an amino acid sequence as shown in SEQ ID NO: 19 and a V_{L} as shown in SEQ ID NO: 23 and a linker separating the scFvs comprising the sequence shown in SEQ ID NO: 27.

In another example, the binding protein may be a di-scFv comprising a V_{H} which comprises an amino acid sequence as shown in SEQ ID NO: 20 and a V_{L} as shown in SEQ ID NO: 24 or a variant thereof that competes for binding to DNA with an antibody which comprises a V_{H} which comprises an amino acid sequence as shown in SEQ ID NO: 20 and a V_{L} as shown in SEQ ID NO: 24 and a linker separating the scFvs comprising the sequence shown in SEQ ID NO: 27.

In another example, the binding protein may be a di-scFv comprising a V_{H} which comprises an amino acid sequence as shown in SEQ ID NO: 21 and a V_{L} as shown in SEQ ID NO: 25 or a variant thereof that competes for binding to DNA with an antibody which comprises a V_{H} which comprises an amino acid sequence as shown in SEQ ID NO: 21 and a V_{L} as shown in SEQ ID NO: 25 and a linker separating the scFvs comprising the sequence shown in SEQ ID NO: 27.

In another example, the binding protein is a di-scFv which comprises an amino acid sequence as shown in SEQ ID NO: 38.

In another example, the Fv is a tri-scFv.

In another example, the scFv, di-scFv or tri-scFv can be linked to a constant region of an antibody, Fc or a heavy chain constant domain C_{H}2 and/or C_{H}3. In one example, the scFv, di-scFv or tri-scFv is linked to the a constant region of an antibody, Fc or a heavy chain constant domain C_{H}2 and/or C_{H}3 by a hinge region. In one example, the hinge region comprises a sequence as shown in SEQ ID NO: 28.

In an example, the binding protein is a di-scFv having an antigen binding domain, wherein the antigen binding domain binds to or specifically binds to DNA.

In another example, the V_{H} and V_{L} of the binding protein are in a separate polypeptide chain. For example, the binding protein can be a diabody, triabody, tetrabody, Fab, F(ab′)₂. In another example, the binding protein can be an Fv which comprises a V_{H} and V_{L} in separate polypeptide chains. In these examples, the binding proteins may be linked to a constant region of an antibody, Fc or a heavy chain constant domain C_{H}2 and/or C_{H}3. In another example, the binding protein can be an intact antibody. Accordingly, in an example, the present disclosure encompasses an antibody having an antigen binding domain, wherein the antigen binding domain binds to or specifically binds to DNA. For example, the antibody comprises a V_{H} comprising an amino acid sequence as shown in SEQ ID NO: 7 and a V_{L} comprising an amino acid sequence as shown in SEQ ID NO: 8. In another example, the antibody comprises a V_{H} comprising an amino acid sequence as shown in SEQ ID NO: 18 and a V_{L} comprising an amino acid sequence as shown in SEQ ID NO: 22 or a variant thereof that competes for binding to DNA with an antibody which comprises a V_{H} which comprises an amino acid sequence as shown in SEQ ID NO: 18 and a V_{L} as shown in SEQ ID NO: 22.

In another example, the antibody comprises a V_{H} comprising an amino acid sequence as shown in SEQ ID NO: 19 and a V_{L} comprising an amino acid sequence as shown in SEQ ID NO: 23 or a variant thereof that competes for binding to DNA with an antibody which comprises a V_{H} which comprises an amino acid sequence as shown in SEQ ID NO: 19 and a V_{L} as shown in SEQ ID NO: 23.

In another example, the antibody comprises a V_{H} comprising an amino acid sequence as shown in SEQ ID NO: 20 and a V_{L} comprising an amino acid sequence as shown in SEQ ID NO: 24 or a variant thereof that competes for binding to DNA with an antibody which comprises a V_{H} which comprises an amino acid sequence as shown in SEQ ID NO: 20 and a V_{L} as shown in SEQ ID NO: 24.

In another example, the antibody comprises a V_{H} comprising an amino acid sequence as shown in SEQ ID NO: 21 and a V_{L} comprising an amino acid sequence as shown in SEQ ID NO: 25 or a variant thereof that competes for binding to DNA with an antibody which comprises a VH which comprises an amino acid sequence as shown in SEQ ID NO: 21 and a VL as shown in SEQ ID NO: 25.

In another example, the antibody is an intact antibody comprising a V_{H} having a CDR1 as shown in SEQ ID NO: 1, a CDR2 as shown in SEQ ID NO: 2, a CDR3 as shown in SEQ ID NO: 3 and a V_{L} having a CDR 1 as shown in SEQ ID NO: 4, a CDR2 as shown in SEQ ID NO: 5 and a CDR3 as shown in SEQ ID NO: 6 or a humanized form thereof. In an example, the antibody is a chimeric antibody. In an example, the V_{H} CDR1 may rather comprise SEQ ID NO: 40.

In another example, the antibody is an intact antibody comprising a V_{H} having a CDR1 as shown in SEQ ID NO: 9 or SEQ ID NO: 10, a CDR2 as shown in SEQ ID NO: 11 or SEQ ID NO: 12, a CDR3 as shown in SEQ ID NO: 13 and a VL having a CDR1 as shown in SEQ ID NO: 14 or SEQ ID NO: 15, a CDR2 as shown in SEQ ID NO: 16 and a CDR3 as shown in SEQ ID NO: 17 or a variant thereof that competes for binding to DNA with an antibody which comprises a VH having a CDR1 as shown in SEQ ID NO: 9 or SEQ ID NO: 10, a CDR2 as shown in SEQ ID NO: 11 or SEQ ID NO: 12, a CDR3 as shown in SEQ ID NO: 13 and a VL having a CDR1 as shown in SEQ ID NO: 14 or SEQ ID NO: 15, a CDR2 as shown in SEQ ID NO: 16 and a CDR3 as shown in

SEQ ID NO: 17. The C_{H}1 and C_{H}2 domains of the antibody may be linked by a hinge region. In one example, the hinge region comprises a sequence as shown in SEQ ID NO: 28.

In another example, the antibody is an intact antibody comprising a V_{H} having a CDR1 as shown in SEQ ID NO: 9, a CDR2 as shown in SEQ ID NO: 11, a CDR3 as shown in SEQ ID NO: 13 and a V_{L} having a CDR1 as shown in SEQ ID NO: 14, a CDR2 as shown in SEQ ID NO: 16 and a CDR3 as shown in SEQ ID NO: 17 or a variant thereof that competes for binding to DNA with an antibody which comprises a V_{H} having a CDR1 as shown in SEQ ID NO: 9, a CDR2 as shown in SEQ ID NO: 11, a CDR3 as shown in SEQ ID NO: 13 and a V_{L} having a CDR1 as shown in SEQ ID NO: 14, a CDR2 as shown in SEQ ID NO: 16 and a CDR3 as shown in SEQ ID NO: 17.

In another example, the antibody is an intact antibody comprising a V_{H} having a CDR1 as shown in SEQ ID NO: 10, a CDR2 as shown in SEQ ID NO: 12, a CDR3 as shown in SEQ ID NO: 13 and a V_{L} having a CDR1 as shown in SEQ ID NO: 15, a CDR2 as shown in SEQ ID NO: 16 and a CDR3 as shown in SEQ ID NO: 17 or a variant thereof that competes for binding to DNA with an antibody which comprises a V_{H} having a CDR1 as shown in SEQ ID NO: 10, a CDR2 as shown in SEQ ID NO: 12, a CDR3 as shown in SEQ ID NO: 13 and a V_{L} having a CDR1 as shown in SEQ ID NO: 15, a CDR2 as shown in SEQ ID NO: 16 and a CDR3 as shown in SEQ ID NO: 17.

In one example, the antibody is an intact antibody comprising a V_{H} which comprises an amino acid sequence as shown in SEQ ID NO: 18 and a V_{L} as shown in SEQ ID NO: 22 or a variant thereof that competes for binding to DNA with an antibody which comprises a V_{H} which comprises an amino acid sequence as shown in SEQ ID NO: 18 and a V_{L} as shown in SEQ ID NO: 22.

In one example, the antibody is an intact antibody comprising a V_{H} which comprises an amino acid sequence as shown in SEQ ID NO: 19 and a V_{L} as shown in SEQ ID NO: 23 or a variant thereof that competes for binding to DNA with an antibody which comprises a V_{H} which comprises an amino acid sequence as shown in SEQ ID NO: 19 and a V_{L} as shown in SEQ ID NO: 23.

In one example, the antibody is an intact antibody comprising a V_{H} which comprises an amino acid sequence as shown in SEQ ID NO: 20 and a V_{L} as shown in SEQ ID NO: 24 or a variant thereof that competes for binding to DNA with an antibody which comprises a V_{H} which comprises an amino acid sequence as shown in SEQ ID NO: 20 and a V_{L} as shown in SEQ ID NO: 24.

In one example, the antibody is an intact antibody comprising a V_{H} which comprises an amino acid sequence as shown in SEQ ID NO: 21 and a V_{L} as shown in SEQ ID NO: 25 or a variant thereof that competes for binding to DNA with an antibody which comprises a V_{H} which comprises an amino acid sequence as shown in SEQ ID NO: 21 and a V_{L} as shown in SEQ ID NO: 25.

In one example, the antibody is an intact antibody comprising the amino acid sequence shown in SEQ ID NO: 39.

As known in the art, antibodies can come in different isotypes such as IgA, IgD, IgE, IgG, and IgM. In one example, antibodies encompassed by the present disclosure are IgG.

In an example, the binding protein is a “nucleolytic binding protein” such as an nucleolytic antibody. These binding proteins can bring about and catalyse cleavage of nucleic acids, such as RNA or DNA. Nucleolytic binding proteins can recognize and interact with DNA or RNA to bring about cleavage of nucleotide-nucleotide linkages at, or near to the region of contact with the DNA or RNA. Accordingly, in certain examples, binding proteins of the disclosure can have “nucleolytic” activity. The term “nucleolytic” is used in this context to refer to a binding protein that can cleave the nucleotide-nucleotide linkages between nucleic acids, for example, by hydrolysis. In an example, the binding protein comprises a V_{H} having an amino acid sequence as shown in SEQ ID NO: 30 and a V_{L} having an amino acid sequence as shown in SEQ ID NO: 34 or a variant thereof that competes for binding to DNA with an antibody which comprises a V_{H} having an amino acid sequence as shown in SEQ ID NO: 30 and a V_{L} having an amino acid sequence as shown in SEQ ID NO: 34. In an example, the antibody comprises a V_{H} having a CDR1 as shown in SEQ ID NO: 31, a CDR2 as shown in SEQ ID NO: 32, a CDR3 as shown in SEQ ID NO: 33 and a V_{L} having a CDR1 as shown in SEQ ID NO: 35, a CDR2 as shown in SEQ ID NO: 36 and a CDR3 as shown in SEQ ID NO: 37 or a variant thereof that competes for binding to DNA with an antibody which comprises a V_{H} having a CDR1 as shown in SEQ ID NO: 31, a CDR2 as shown in SEQ ID NO: 32, a CDR3 as shown in SEQ ID NO: 33 and a V_{L} having a CDR1 as shown in SEQ ID NO: 35, a CDR2 as shown in SEQ ID NO: 36 and a CDR3 as shown in SEQ ID NO: 37.

An example, of a nucleolytic antibody is disclosed in Noble et al. (2014) Sci Rep-Uk., 4: 5958.

In another example, binding proteins of the disclosure can bind DNA without facilitating degradation of the DNA.

In an example, the antibody is monoclonal. Monoclonal antibodies are one exemplary form of antibodies contemplated by the present disclosure. The term “monoclonal antibody” or “MAb” refers to a homogeneous antibody population capable of binding to the same antigen(s), for example, to the same epitope within the antigen. This term is not intended to be limited as regards to the source of the antibody or the manner in which it is made.

In an example, antibodies encompassed by the present disclosure may be “humanized”. In an example, the CDRs are humanized. A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. In an example, the humanized antibody will also comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). In an example, antibodies of the disclosure are used to deliver a payload to the brain of a subject.

Binding proteins can be identified by their ability to compete with a reference binding protein for binding to DNA using various methods known in the art. For example, an anti-DNA binding protein of the disclosure is conjugated with biotin using established procedures (Hofmann K, et al. (1982) Biochemistry 21: 978-84). Candidate binding proteins are then evaluated by their capacity to compete with the binding of the biotinylated antibody to DNA. The binding of biotinylated antibody to DNA may be assessed by the addition of fluorescein-labelled streptavidin which will bind to biotin on the labelled binding protein. Fluorescence staining is then quantified, and the competitive effect of binding protein(s) expressed as a percentage of the fluorescence levels obtained in the absence of the candidate competitor. In other examples, affinity measurements are used to determine the competitive effect of candidate binding proteins. Affinity measurements can be determined by standard methodology for antibody reactions, for example, immunoassays, surface plasmon resonance (SPR) (Rich and Myszka Curr. Opin. Biotechnol 11:54, 2000; Englebienne Analyst. 123: 1599, 1998), isothermal titration calorimetry (ITC) or other kinetic interaction assays known in the art. In one example, the constants are measured by using surface plasmon resonance assays, e.g., using BIAcore surface plasmon resonance (BIAcore, Inc., Piscataway, NJ) with immobilized DNA. Exemplary SPR methods are described in U.S. Pat. No. 7,229,619.

### Antibody Fragments

Single Chain Fv (scFv) Fragments

One of skill in the art will be aware that scFv's comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). Single-chain variable fragments lack the constant Fc region found in complete antibody molecules and therefore can have reduced immunogenicity. Exemplary linkers comprise in excess of 12 amino acid residues with (Gly₄Ser)₃ being one of the more favoured linkers for a scFv. Another example of a suitable linker is provided in SEQ ID NO: 26.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

In another example, the present disclosure encompasses a dimeric scFv (di-scFV), i.e., a protein comprising two scFv molecules linked by a non-covalent or covalent linkage, e.g., by a leucine zipper domain (e.g., derived from Fos or Jun) or trimeric scFV (tri-scFv). In another example, two scFv's are linked by a peptide linker of sufficient length to permit both scFv's to form and to bind to an antigen, e.g., as described in U.S. Published Application Ser. No. 20060263367. An exemplary linker is provided in SEQ ID NO: 27.

### Diabodies, Triabodies, Tetrabodies

In some examples, an antigen binding fragment of the disclosure is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} Or V_{H}-X-V_{L}, wherein X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fv's having different specificity).

### Other Antibodies and Antibody Fragments

Other examples of antibodies encompassed by the present disclosure include:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab₃ (e.g., as described in EP19930302894).

### Binding Protein Production

### Recombinant Expression

In an example, the binding protein is recombinant.

In the case of a recombinant binding protein such as an antibody or fragment thereof, a nucleic acid encoding the same can be cloned into expression vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce immunoglobulin or antibody protein.

Suitable molecular cloning techniques are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art. See U.S. Pat. No. 4,816,567 or U.S. Pat. No. 5,530,101.

Following isolation, the nucleic acid is operably linked to a promoter in an expression construct or expression vector for further cloning (amplification of the DNA) or for expression in a cell-free system or in cells. Thus, another example of the disclosure provides an expression construct that comprises an isolated nucleic acid encoding a binding protein of the disclosure and one or more additional nucleotide sequences. Suitably, the expression construct is in the form of, or comprises genetic components of, a plasmid, bacteriophage, a cosmid, a yeast or bacterial artificial chromosome as are understood in the art. Expression constructs may be suitable for maintenance and propagation of the isolated nucleic acid in bacteria or other host cells, for manipulation by recombinant DNA technology and/or for expression of the nucleic acid encoding a binding protein of the disclosure.

Many vectors for expression in cells are available. The vector components generally include, but are not limited to, one or more of the following: a signal sequence (e.g. SEQ ID NO: 29), a sequence encoding the binding protein (e.g., derived from the amino acid sequence information provided herein), an enhancer element, a promoter, and a transcription termination sequence. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin or antibody promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the binding protein may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

The skilled artisan will understand from the foregoing description that the present disclosure also provides an isolated nucleic acid encoding a binding protein of the present disclosure.

The present disclosure also provides an expression construct comprising an isolated nucleic acid of the disclosure operably linked to a promoter. In one example, the expression construct is an expression vector.

In one example, the expression construct of the disclosure comprises a nucleic acid encoding a polypeptide (e.g., comprising a V_{H}) operably linked to a promoter and a nucleic acid encoding another polypeptide (e.g., comprising a V_{L}) operably linked to a promoter.

The disclosure also provides a host cell comprising an expression construct according to the present disclosure.

The present disclosure also provides an isolated cell expressing a binding protein of the disclosure or a recombinant cell genetically-modified to express the binding protein.

Methods for purifying antibodies according to the present disclosure are known in the art and/or described in publications such as WO2019/018426.

### Compositions

The present disclosure includes pharmaceutical compositions for administration to subjects. Exemplary compositions comprise one or more of the above referenced binding proteins.

The compositions can also contain a pharmaceutically acceptable carrier or adjuvant for administration of the binding protein. In some embodiments, the carrier is pharmaceutically acceptable for use in humans. The carrier or adjuvant should not itself induce the production of antibodies harmful to the individual receiving the composition and should not be toxic. Suitable carriers can be large, slowly metabolized macromolecules such as proteins, polypeptides, liposomes, polysaccharides, polylactic acids, polyglycolic acids, polymeric amino acids, ammo acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonate and benzoates.

Pharmaceutically acceptable carriers in therapeutic compositions can additionally contain liquids such as water, saline, glycerol and ethanol. Additionally, auxiliary substances, such as wetting or emulsifying agents or pH buffering substances, can be present in such compositions.

The compositions of the presently disclosed subject matter can further comprise a carrier to facilitate composition preparation and administration. Any suitable delivery vehicle or carrier can be used, including but not limited to a microcapsule, for example a microsphere or a nanosphere (Manome et al. (1994) Cancer Res 54:5408-5413; Saltzman & Fung (1997) Adv Drug Deliv Rev 26:209-230), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate (Goldman et al. (1997) Cancer Res 57: 1447-1451 and U.S. Pat. Nos. 4,551,482, 5,714,166, 5,510,103, 5,490,840, and 5,855,900), and a polysome (U.S. Pat. No. 5,922,545).

A composition of the present invention may comprise a pharmaceutical composition that includes a pharmaceutically acceptable carrier. Suitable formulations include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS in the range of 0.1 to 10 mg/ml, about 2.0 mg/ml; and/or mannitol or another sugar in the range of 10 to 100 mg/ml, in some embodiments about 30 mg/ml; and/or phosphate-buffered saline (PBS). Any other agents conventional in the art having regard to the type of formulation in question can be used. In some examples, the carrier is pharmaceutically acceptable. In some examples, the carrier is pharmaceutically acceptable for use in humans.

Compositions of the present disclosure can have a pH between 5.5 and 8.5, preferably between 6 and 8, and more preferably about 7. The pH can be maintained by the use of a buffer. The composition can be sterile and/or pyrogen free. The composition can be isotonic with respect to humans. Compositions of the presently disclosed subject matter can be supplied in hermetically-sealed containers.

The compositions can include an effective amount of one or more binding proteins as described herein. In some embodiments, a pharmaceutical composition can comprise an amount that is sufficient to treat, ameliorate, or prevent a desired disease or condition, or to exhibit a detectable therapeutic effect. For example, in some examples, the composition includes an effective amount of one or more binding proteins to reduce Neutrophil Extracellular Trap (NET) formation or NETosis under culture conditions or in a subject in need thereof, e.g., relative to a control. In some examples, the composition includes an effective amount of one or more binding proteins to reduce Neutrophil

Extracellular Trap (NET) formation or NETosis in an effective amount to reduce or prevent one or more symptoms of disease or disorder.

Binding proteins of the disclosure and compositions comprising the same can be administered in a variety of unit dosage forms depending upon the method of administration. Dosages for typical binding protein such as antibody or fragment pharmaceutical compositions are well known to those of skill in the art. Such dosages are typically advisory in nature and are adjusted depending on the particular therapeutic context or patient tolerance. The amount binding protein adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton, Peptides 18: 1431-1439, 1997; Langer, Science 249: 1527-1533, 1990.

Routes of administration include, but are not limited to, injection, subcutaneous, intramuscular, intraarticular, intravenous, intraarterial. In one example, the binding protein is delivered through intravenous administration. In another example, the binding protein is delivered through subcutaneous administration. In another example, the binding protein is delivered through injection. In another example, the binding protein is delivered through infusion. In another example, the binding protein is delivered through intraarticular injection into a joint space. In another example, the binding protein is delivered through intraarticular injection into an arthritic joint.

The compositions can be administered in a single dose treatment or in multiple dose treatments on a schedule and over a time period appropriate to the age, weight and condition of the subject, the particular binding protein formulation used, and the route of administration.

### Conditions to be treated, diagnosed or monitored

The methods of the disclosure encompass treatment, prophylaxis, imaging and diagnosis of various inflammatory diseases. As used herein, the term, “inflammatory disease” should be taken to encompass diseases associated with NET formation or NETosis. In an example, the diseases are associated with increased NET formation or NETosis. Exemplary inflammatory diseases include cystic fibrosis, ischemia, cardiovascular disease, periodontitis, fibrosis, pruritus, skin inflammation, psoriasis, multiple sclerosis, rheumatoid arthritis, osteoarthritis, systemic lupus erythematosus, Hashimoto's thyroidis, myasthenia gravis, diabetes type I or II, diabetic nephropathy, asthma, inflammatory liver injury, inflammatory glomerular injury, atopic dermatitis, allergic contact dermatitis, irritant contact dermatitis, seborrhoeic dermatitis, Sjoegren's syndrome, keratoconjunctivitis, uveitis, vasculitis, inflammatory bowel disease, Crohn's disease, ulcerative colitis, acute or chronic idiopathic inflammatory arthritis, myositis, a demyelinating disease, chronic obstructive pulmonary disease, interstitial lung disease, interstitial nephritis, chronic active hepatitis, gout, metabolic disease, inflammation associated with obesity, delayed wound healing, trauma, sepsis, septic joint, septic arthritis, aseptic arthritis.

For example, the inflammatory disorder can be delayed wound healing. In an example, the delayed wound healing is secondary to diabetes. Examples of metabolic disease include diabetes and obesity.

In an example, the inflammatory disease is an autoimmune disease. Examples of autoimmune diseases include lupus, rheumatoid arthritis and ANCA associated vasculitis (AAV). In an example, the inflammatory disease is vasculitis. For example, the inflammatory disease can be ANCA associated vasculitis (AAV). AAV is an umbrella term for a group of multi-system autoimmune small vessel vasculitides. AAV diseases include microscopic polyangiitis, granulomatosis with polyangiitis (GPA, previously “Wegener's granulomatosis”), and eosinophilic granulomatosis with polyangiitis (EGPA, previously “Churg-Strauss syndrome”). In an example, treatment of vasculitis is characterised by reduced segmental necrosis. For example, the percentage of glomeruli with segmental necrosis reduces after treatment. In an example, treatment of vasculitis is characterised by reduced glomerular infiltration. In an example, glomerular macrophage levels decrease after treatment. In another example, glomerular neutrophil levels decrease after treatment.

In another example, the inflammatory disease is selected from the group consisting of pneumonia, neutrophilic asthma, neutrophil-mediated anaphylaxis, inflammatory lung injury, chronic obstructive pulmonary disease (COPD).

In another example, the inflammatory disease is Acute Respiratory Distress Syndrome (ARDS). In an example, the ARDS is moderate or severe.

In another example, the inflammatory disease is caused by a viral infection such as a rhinovirus, an influenza virus, a respiratory syncytial virus (RSV) or a coronavirus. In an example, the viral infection is caused by a coronavirus. In an example, the coronavirus is severe acute respiratory syndrome coronavirus (SARS-CoV), Middle East respiratory syndrome coronavirus (MERS-CoV) or severe acute respiratory syndrome coronavirus 2 (SARS-CoV-2).

In a particular example, the inflammatory disease is coronavirus disease 19 (COVID-19).

In an example, the inflammatory disease is graft versus host disease. In another example, the inflammatory disease is fibrosis. Examples include lung and kidney fibrosis.

In an example, the inflammatory disease is glomerulonephritis.

In an example, the inflammatory disease is sepsis.

In an example, the inflammatory disease is septic or aseptic arthritis.

In an example, the inflammatory disease is diffuse intravascular coagulation (DIC).

In an example, the inflammatory disease is a thrombosis or a thrombo-inflammatory disease such as venous embolism, disseminated intravascular coagulation, ischemia/reperfusion injury, myocardial infarction or stroke.

In an example, the inflammatory disease is a neurodegenerative disorder. For example, the inflammatory disease can be multiple sclerosis. In an example, the inflammatory disease is Alzheimer's disease.

### Methods of Treatment and Methods of Prophylaxis

Various therapeutic and prophylactic applications are envisaged in view of the findings indicating that binding proteins of the disclosure can inhibit NET formation. In an example, the present disclosure encompasses methods of treatment or prophylaxis which comprise administering the binding proteins or compositions described herein to a subject in need thereof. In an example, the subject has an inflammatory disease.

Accordingly, in an example, the present disclosure provides a method of treating an inflammatory disease in a subject, comprising administering to the subject, a binding protein or composition disclosed herein.

In one example, the present disclosure provides a method of treating an inflammatory disease in a subject, comprising administering a binding protein comprising a VH having a CDR1 as shown in SEQ ID NO: 1, a CDR2 as shown in SEQ ID NO: 2, a CDR3 as shown in SEQ ID NO: 3 and a VL having a CDR 1 as shown in SEQ ID NO: 4, a CDR2 as shown in SEQ ID NO: 5 and a CDR3 as shown in SEQ ID NO: 6 or a humanized form thereof. In an example, the V_{H} CDR1 may rather comprise SEQ ID NO: 40. For example, the method can comprise administering a binding protein comprising a VH having a CDR1 as shown in SEQ ID NO: 9, a CDR2 as shown in SEQ ID NO: 11, a CDR3 as shown in SEQ ID NO: 13 and a VL having a CDR 1 as shown in SEQ ID NO: 14, a CDR2 as shown in SEQ ID NO: 16 and a CDR3 as shown in SEQ ID NO: 17.

In another example, the present disclosure provides a method of treating an inflammatory disease in a subject, comprising administering a binding protein comprising a VH comprising an amino acid sequence as shown in SEQ ID NO: 18 and a VL comprising an amino acid sequence as shown in SEQ ID NO: 22.

In another example, the present disclosure provides a method of treating an inflammatory disease in a subject, comprising administering a binding protein comprising a VH which comprises an amino acid sequence as shown in SEQ ID NO: 7 and a VL as shown in SEQ ID NO: 8 or a humanized form thereof.

In another example, treatment comprises administering a nucleolytic binding protein disclosed herein.

Prophylactic methods of the disclosure encompass administering to a subject, a cell penetrating, anti-DNA binding protein disclosed herein to prevent NET formation in the subject or reduce NET formation in the subject. In an example, the subject has an inflammatory disease. In another example, the subject is at risk of developing an inflammatory disease. For example, the subject may be in remission from an inflammatory disease or may have sustained an injury or infection or exposure that is expected to cause NETosis. Accordingly, in an example, the present disclosure encompasses methods of preventing Neutrophil Extracellular Trap (NET) formation in a subject, the method comprising administering to the subject a binding protein disclosed herein, wherein the binding protein inhibits NET formation under culture conditions. In another example, the present disclosure encompasses a method of inhibiting Neutrophil Extracellular Trap (NET) formation in a subject, the method comprising administering to the subject a cell penetrating, anti-DNA binding protein, wherein the cell penetrating, anti-DNA binding protein inhibits NET formation under culture conditions.

In an example, subjects treated according to the disclosure have an increased risk of thrombosis or embolism. For example, a subject's inflammatory disorder may increase their risk of thrombosis or embolism. In another example, the subject is infected with a virus that increases their risk of thrombosis or embolism. For example, the subject may have a coronavirus infection.

In another example, the present disclosure provides the use of a binding protein or composition disclosed herein in the manufacture of a medicament for treating an inflammatory disease in a subject.

In view of the contribution by NETs the formation of thrombosis or embolism, in another example, the methods of the present disclosure encompass treatment or prevention of these events by administering a binding protein disclosed herein. In an example, the methods of the disclosure encompass a method of treating or preventing thrombosis or embolism in a subject, the method comprising administering to the subject a cell penetrating, anti-DNA binding protein, wherein the cell penetrating, anti-DNA antibody inhibits Neutrophil Extracellular Trap (NET) formation under culture conditions. In another example, a binding protein of the disclosure can be administered to a subject prophylactically to reduce the risk of thrombosis or embolism.

### EXAMPLES

### Example 1—DX1 Penetrates PLB-985 cells

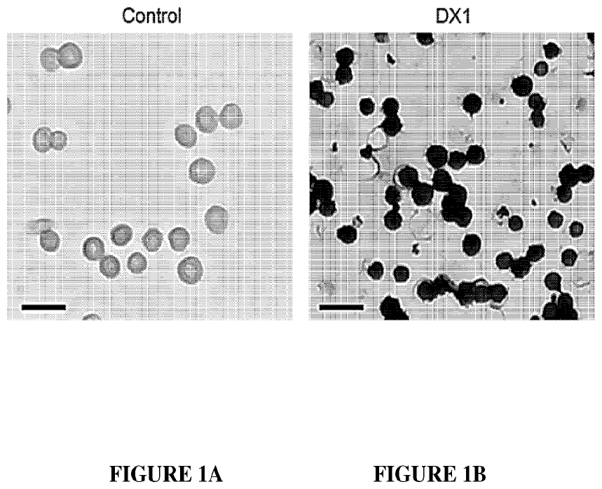

Work with isolated mouse or human neutrophils is challenging due to their short functional lifespans, and granulocyte-differentiated HL-60 and their subclone PLB-985 human acute myeloid leukemia cells provide a reliable alternative to isolated neutrophils for NETosis studies. The nuclear-penetrating anti-DNA autoantibody 3E10 and its derivative DX1 require expression of the nucleoside transporter ENT2 in order to penetrate target cells. ENT2 expression in HL-60 cells has previously been reported, and DX1 was confirmed to penetrate into ˜100% of PLB-985 cells in culture (FIG. 1).

### Example 2—DX1 Inhibits NET Formation by Neutrophil-Like Cells

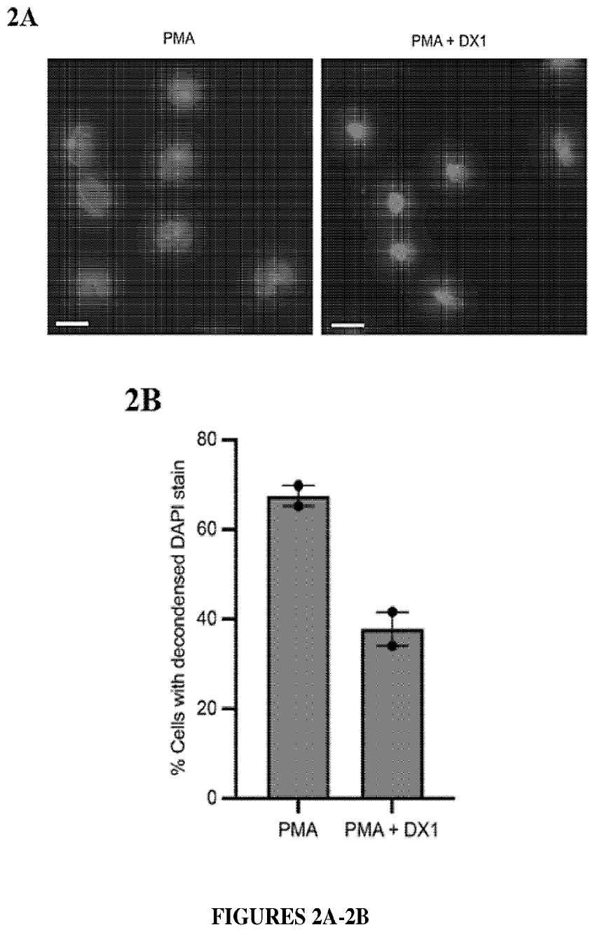

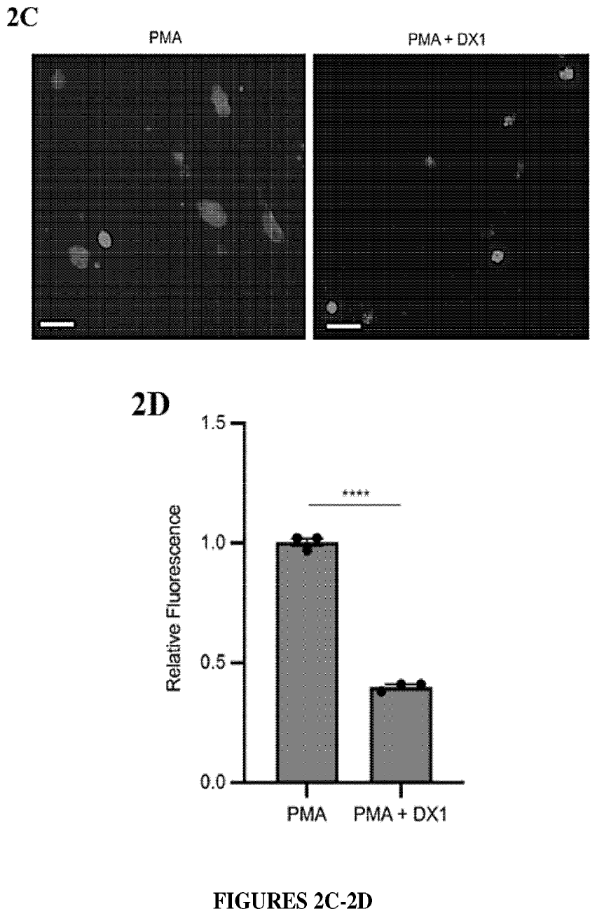

Neutrophils treated with PMA undergo a cascade triggered by NADPH oxidase (NOX)-mediated production of reactive oxygen species (ROS) that ultimately results in the release of NETs through NOX-dependent NETosis. Differentiated PLB-985/HL-60 cells mirror the activity of neutrophils and respond to PMA stimulation by decondensing DNA and releasing NETs, and their chromatin decondensation can be visualized by DAPI staining. Differentiated PLB-985 cells were treated with control media or media containing 10 μM DX1 for one hour, followed by addition of PMA and subsequent visualization of chromatin status by DAPI stain. The diffuse DAPI stain suggestive of chromatin decondensation was observed in 67.5±1.6% of control cells and 37.8±2.7% of cells pre-treated with DX1 (N=2) (FIG. 2A, B).

SYTOX™ Green is an impermeant fluorescent DNA stain that facilitates visualization of DNA released in NETosis. Differentiated PLB-985 cells treated with control or 10 μM DX1 and stimulated with PMA were exposed to SYTOX™ Green, and fluorescence was visualized under fluorescence microscopy and quantified by plate reader. DX1 significantly reduced the resulting SYTOX™ Green signal, with fluorescence reduced to 0.40±0.01 relative to control (P<0.0001, N=3) (FIG. 2C, 2D). This result is consistent with inhibition of DNA release mediated by DX1.

The results of the DAPI and SYTOX™ Green staining studies in FIG. 2C, 2D are consistent with DX1-mediated inhibition of chromatin decondensation and DNA release by differentiated PLB-985 cells. To further probe this effect, DNA-protein release was measured by differentiated PLB-985 cells after PMA stimulation by resuspending and quantifying DNA-protein complexes adherent to wells by spectrophotometry using a previously described protocol for isolation and quantification of NET-DNA in cell culture. This assay is based on the concept that intact cells for the most part remain in suspension, while DNA-protein complexes released in NETs are at least partially adhered to wells. This distinction allows for separation of intact cells from adherent NET-DNA by aspiration of the cell culture supernatants, followed by resuspension of adherent DNA-protein complexes and quantification by spectrophotometry.

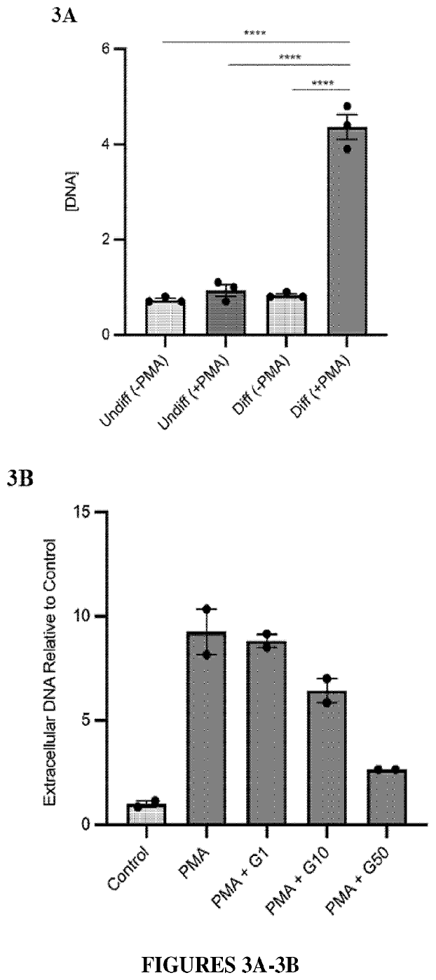

A control study was conducted to confirm differential response of the undifferentiated and differentiated PLB-985 cells to PMA in this assay. Undifferentiated and differentiated PLB-985 cells were treated with control or 300 nM PMA followed by quantification of the expected NET-DNA adherent to wells by spectrophotometry. Control and PMA-stimulated undifferentiated PLB-985 cells yielded similar DNA concentrations of 0.73±0.03 and 0.93±0.12 ng/μL (P=0.12, N=3), respectively, consistent with the expected absence of response by undifferentiated cells to PMA. In contrast, control and PMA-stimulated differentiated PLB-985 cells yielded DNA concentrations of 0.83±0.03 and 4.40±0.27 ng/μL (P=0.002, N=3), consistent with the expected release of DNA in response to PMA (FIG. 3A). These findings support the conclusions that differentiated PLB-985 cells exhibited granulocyte-like behavior with response to PMA measurable by the reported spectrophotometry method. Further, the absence of any apparent increase in DNA release by undifferentiated PLB-985 cells treated with PMA rules out any major contribution from nonspecific PMA toxicity to the measured DNA concentrations.

Next, the ability of this assay to detect the effect of a known NETosis inhibitor on DNA release by differentiated PLB-985 cells stimulated by PMA was verified. The activity of peptidylarginine deiminase 4 (PAD4) in catalyzing histone citrullination is important to granulocyte NETosis, and the small molecule selective PAD4 inhibitor GSK484 has previously been shown to inhibit NET release. Differentiated PLB-985 cells were treated with 0-50 μM of GSK484 for one hour prior to stimulation with PMA, followed by quantification of adherent DNA by spectrophotometry. GSK484 reduced extracellular DNA in a dose-dependent manner, consistent with suppression of NETosis in differentiated PLB-985 cells (FIG. 3B).

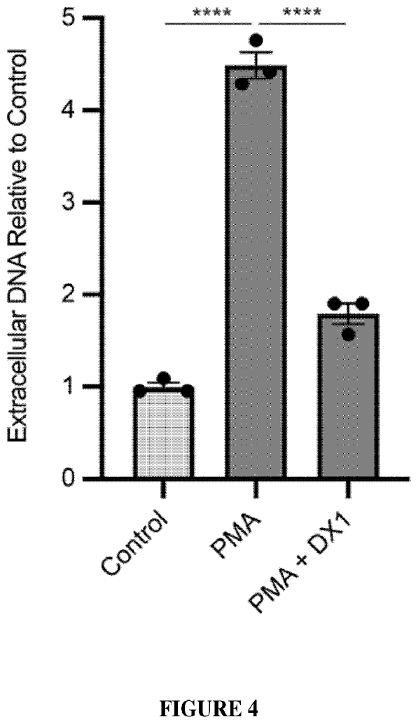

The differential response of undifferentiated and differentiated PLB-985 cells to PMA and the observed inhibition of response to PMA mediated by the known PAD4 inhibitor GSK484 supported the use of the DNA spectrophotometry assay in evaluating the effect of DX1 on NETosis in differentiated PLB-985 cells. Differentiated PLB-985 cells were treated with media containing control buffer or 10 μM DX1 for one hour followed by stimulation by addition of PMA and subsequent quantification of DNA release by spectrophotometry as described above. Results were expressed relative to DNA content obtained in unstimulated cells treated with control media. PMA stimulation increased relative DNA content to 4.50±0.14 (P<0.0001, N=3), while DX1 suppressed the increase in DNA release to 1.80±0.11 (P<0.0001, N=3) (FIG. 4). These results are consistent with inhibition of NETosis by DX1, and the findings of the DAPI and SYTOX™ Green assays described above.

### Example 3—DX1 Does Not Reduce Citrullination of H3 in Neutrophil-Like Cells

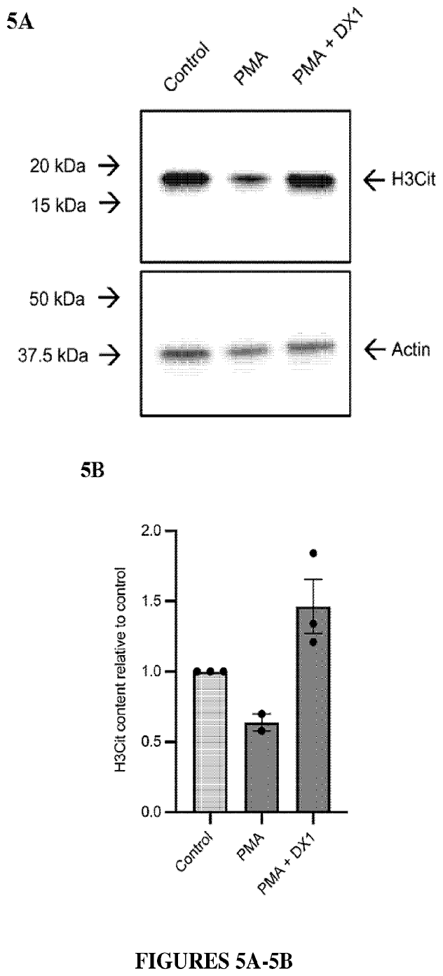

Histone citrullination by PAD4 is a key step in NETosis that is targeted by the selective PAD4 inhibitor GSK484. Differentiated PLB-985 cells treated with control or 10 μM DX1 for one hour were stimulated by PMA, and cell contents were isolated and analyzed for H3Cit content by western blot with normalization to actin. Stimulation of control cells with PMA was associated with a reduction in H3Cit content to 0.64±0.06 relative to unstimulated cells, consistent with the release of H3Cit in the process of NET formation. In contrast, cells treated with DX1 exhibited an apparent increase in H3Cit content to 1.46±0.20 relative to unstimulated cells (FIG. 5A, 5B), possibly indicating retention of H3Cit within the cells due to inhibition of NET release by DX1. These data indicate that DX1 does not interfere with citrullination of H3 but rather inhibits the release of NETs and their associated H3Cit content.

### Example 4—DX1 Does Not Cause NETosis in Mouse Neutrophils

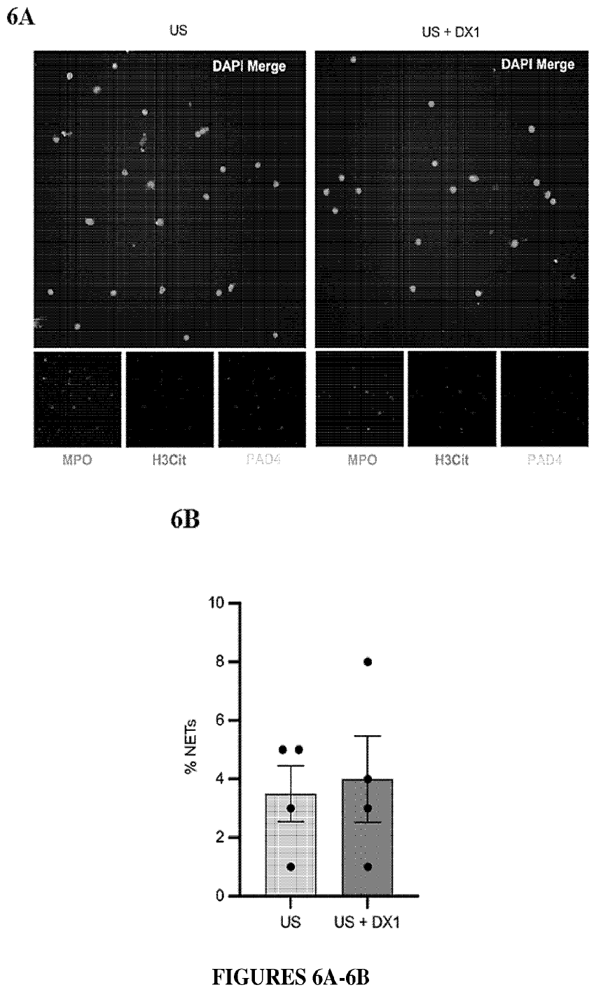

Neutrophils obtained from thioglycollate-treated mice and directly plated onto 20-well plates were treated with control media or media containing 10 μM DX1 for 30 minutes, and then observed for four hours, after which cells were fixed and immunostained for markers of NETs including myeloperoxidase (MPO), H3Cit, and peptidylarginine deiminase 4 (PAD4). The percentage of NETs released per 100 cells was determined by visualization on confocal microscopy and analyzed by ImageJ (NIH). Few NETs were observed in unstimulated neutrophils treated with control or DX1 (3.5%±1.0 and 4.0%±1.5, respectively, P=0.79, N=4) (FIG. 6A, 6B).

### Example 5—DX1 Inhibits NOX-Dependent NETosis in Mouse Neutrophils

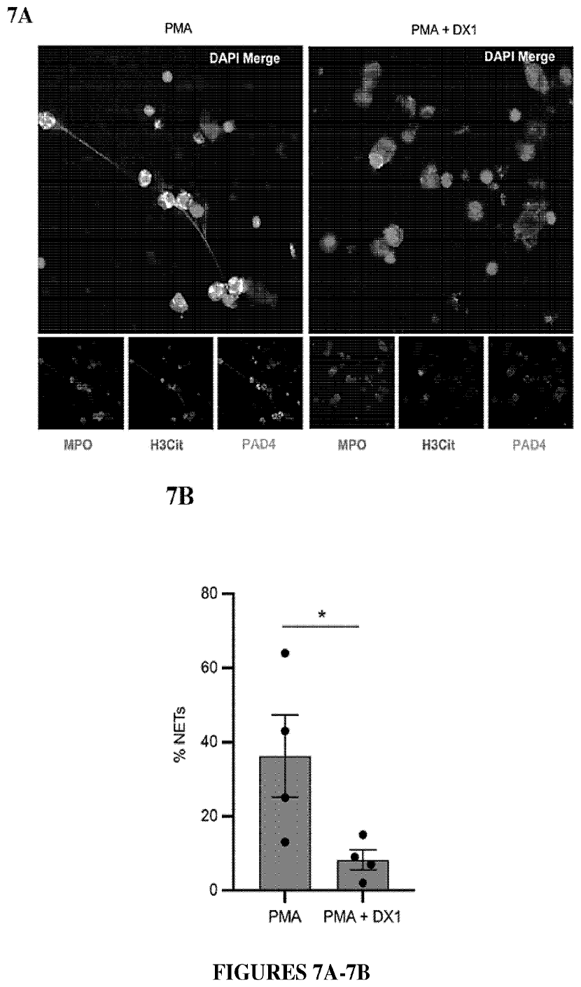

Neutrophils obtained from thioglycollate-treated mice and directly plated onto 20-well plates were treated with control media or media containing 10 μM DX1 for 30 minutes, and then stimulated with PMA for induction of NOX-dependent NETosis for four hours, after which cells were fixed and immunostained for markers of NETs including MPO, H3Cit, and PAD4. The percentage of NETs released per 100 cells was determined by visualization on confocal fluorescence microscopy and analyzed by ImageJ (NIH). PMA stimulation yielded NETs in 36.3%±11.1 of control neutrophils, but only 8.3%±2.7 of DX1-treated neutrophils (P=0.05, N=4) (FIG. 7A, 7B). These results demonstrate inhibition of NOX-dependent NETosis mediated by PMA in mouse neutrophils.

### Example 6—DX1 Inhibits NOX-Dependent NETosis in Mouse Neutrophils

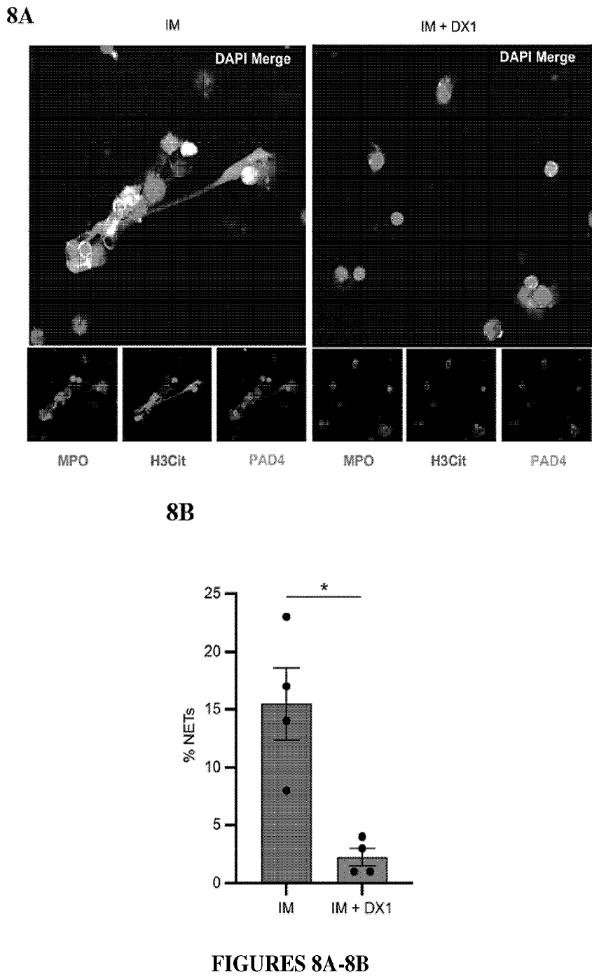

Neutrophils obtained from thioglycollate-treated mice and directly plated onto 20-well plates were treated with control media or media containing 10 μM DX1 for 30 minutes, and then stimulated with a calcium ionophore (ionomycin, IM) for induction of NOX-independent NETosis for four hours, after which cells were fixed and immunostained for markers of NETs including MPO, H3Cit, and PAD4. The percentage of NETs released per 100 cells was determined by visualization on confocal fluorescence microscopy and analyzed by ImageJ (NIH). IM stimulation resulted in NETs in 15.5%±3.1 of control neutrophils, but only 2.3% 35 0.8 in DX1-treated neutrophils (P<0.01, N=4) (FIG. 8A, 8B). These results demonstrate inhibition of NOX-independent mechanisms of NETosis mediated by IM in mouse neutrophils.

### Example 7—DX1 Significantly Reduces Glomerular Inflammation and Pathology in ANCA Associated Vasculitis

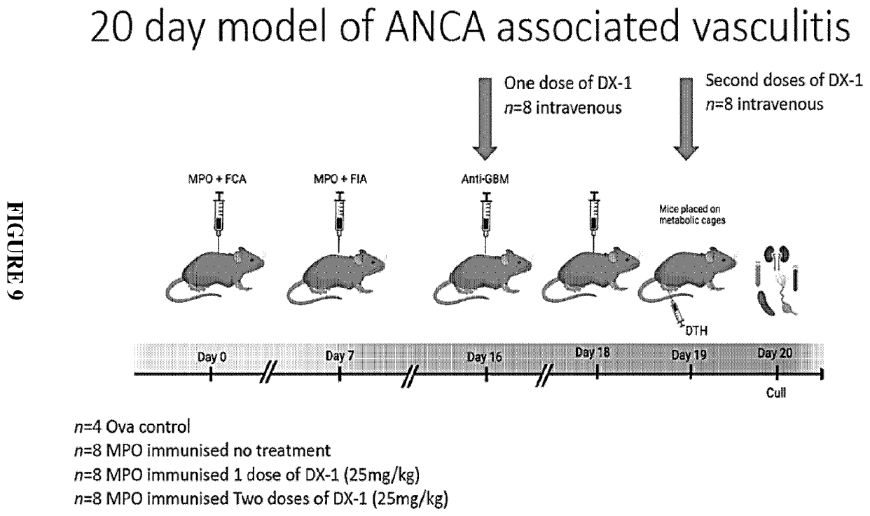

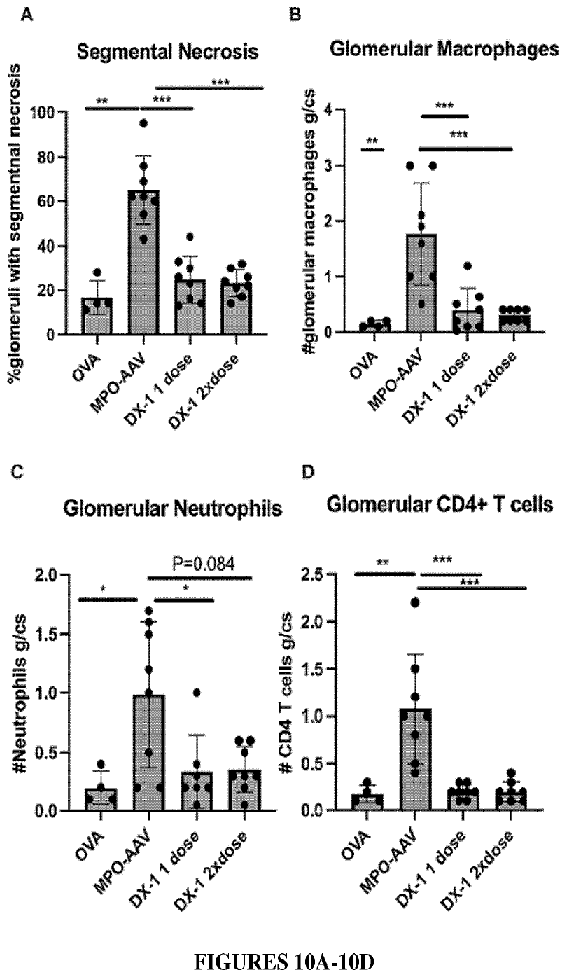

As noted above, NETs contribute to the pathophysiology of multiple disease processes, in particular in the context of inflammatory disease. In view of the above referenced data showing that cell penetrating, anti-DNA binding proteins such as DX1 can penetrate neutrophil and neutrophil-like cells and inhibit NET formation, the functional implications of these findings were confirmed in an animal model of vasculitis (summarized in FIG. 9.). As shown in FIGS. 10A-10D, DX1 significantly reduced glomerular inflammation and pathology in MPO immunized mice that received DX1. DX1 reduced inflammation and pathology to levels corresponding with Ova controls. These data provide further evidence of the prospect of administering anti-DNA binding proteins such as DX1 to inhibit NET formation, in particular in the context of treating inflammatory disease.

The use of cell penetrating, anti-DNA binding proteins such as DX1 is particularly interesting in the context of ANCA vasculitis as the MPO enzyme implicated in NETosis is also a cause of disease with patients developing anti-MPO autoantibodies. Thus, in this disease, use of cell penetrating, anti-DNA binding proteins such as DX1 may be used for prevention and treatment included management of disease progression.

### SUMMARY

The above referenced data show that cell penetrating, anti-DNA binding proteins such as DX1 can penetrate neutrophil and neutrophil-like cells and inhibit NET formation.

As NETs contribute to the pathophysiology of multiple disease processes including trauma, autoimmunity, genetic disease, and cancer, the present findings add a new dimension to these binding proteins. Indeed, methods to modulate NET formation are already of clinical interest, and systemic administration of DNase I or inhibitors of histone citrullination to promote dissolution of NET-DNA or prevent NET formation have been reported (Park et al. (2016) Sci Transl Med., 8: 361ra138; Perdomo et al. (2019) Nat Comm., 10:1322).

Accordingly, the present findings demonstrate potential for cell penetrating, anti-DNA binding proteins such as DX1 in both treatment and prophylaxis of non-malignant conditions in which suppression of NET formation (NETosis) is needed.

NETs also contribute to cancer and metastasis. Thus, the present findings add a new dimension to these molecules in treatment and/or prophylaxis of cancer and/or metastatic disease.

### METHODS

### Reagents and PLB-985 Cells

The 3E10 derivative DX1 (PAT-DX1, Patrys Ltd, Melbourne, Australia) (a di-scFv having the sequence (SEQ ID NO: 38) was generated and purified as previously described (Rattray et al. (2018) Biochem Biophys Res Commun. 496: 858-864; Colburn and Green (2006) Clin Chim Acta. 370: 9-16). GSK484 (#SML1658) was obtained from MilliporeSigma (St. Louis, MO). PLB-985 cells, a subclone of HL-60 human acute myeloid leukemia cells, were obtained by material transfer agreement with the Leibniz Institute DSMZ-German Collection of Microorganisms and Cell Cultures GmbH (Braunschweig, Germany) and used within one month of receipt. Cells were grown in RPMI+10% FBS at 37° C. with 5% CO2, and differentiated to granulocyte-like cells by addition of 1.3% DMSO to the growth medium for 6 days. Unless otherwise specified, all other materials and reagents were obtained from Thermo Fisher Scientific (Waltham, MA, USA).

### Cellular Penetration Assay

PLB-985 cells cultured on Shi-fix coverslips (Shikhar Biotech, Khumaltar, Lalitpur, Nepal) were treated for one hour with control media or media containing 5 μM DX1 and then washed and fixed in 100% chilled ethanol. Presence of DX1 in the cells was evaluated by incubation with Pierce™ Recombinant Protein L (1:1000) (#21189, Thermo Fisher Scientific), protein L polyclonal antibody (1:1000) (#PA1-72066, Thermo Fisher Scientific), and goat anti-chicken IgY (H+L) alkaline phosphatase secondary antibody (1:1000) (#PA1-28799, Thermo Fisher Scientific). Alkaline phosphatase-mediated signal development was visualized under brightfield microscopy (EVOS FL, Thermo Fisher Scientific).

### Visualization of Chromatin Decondensation in Differentiated PLB-985 Cells

Differentiated PLB-985 cells were plated on Shi-fix coverslips in 12-well plates at 5×10⁴ cells/well. Cells were treated with control media or media containing 10 μM DX1 for one hour, after which cells were stimulated by addition of 300 nM PMA for two hours. Cells were fixed with ethanol followed by addition of DAPI and visualization of chromatin decondensation by fluorescence microscopy (EVOS FL, Thermo Fisher Scientific). The percentage of cells that demonstrated the expected response to PMA stimulation was measured by counting the number of total cells and cells with visualized DAPI signal decondensation in a minimum of 150 cells per treatment condition.

### Visualizing and Quantifying Extracellular DNA by SYTOX™ Green Stain

Differentiated PLB-985 cells were cultured in black wall 96-well plates at 5×10⁴ cells/well and incubated with control buffer or 10 μM DX1 for one hour. Cells were then stimulated by addition of 300 nM PMA for two hours. SYTOX™ Green (5 μM) was added to facilitate detection of extracellular DNA. SYTOX™ Green fluorescence was visualized by fluorescence microscopy (EVOS FL, Thermo Fisher Scientific) and fluorescence intensity measured by plate reader at excitation and emission wavelengths 485 nm and 527 nm.

### Quantifying DNA Released by Differentiated PLB-985 Cells After Stimulation With PMA by Spectrophotometry

Differentiated PLB-985 cells were plated at 8×10⁵ cells/well in 12-well plates and incubated with control buffer or buffer containing 10 μM DX1 for one hour. Cells were then stimulated by addition of 300 nM PMA for four hours (stimulation duration increased to four hours compared to DAPI and SYTOX™M Green stain and western blot experiments to allow more time for adherence of DNA-protein complexes to wells). Supernatants were aspirated and wells were gently washed once with PBS. Residual contents adherent to the wells were extracted into PBS and DNA content was determined by spectrophotometry using the Nanodrop Lite (Thermo Fisher Scientific) in accordance with the manufacturer's instructions.

### H3Cit Western Blot

Differentiated PLB-985 cells at 8×10⁵ cells/well in 12-well plates were incubated with control buffer or buffer containing 10 μM DX1 for one hour and then stimulated by addition of 300 nM PMA for two hours. Cells were isolated by centrifugation, lysed in RIPA buffer, and H3Cit content determined by western blot using anti-H3Cit primary (#AB5103, abcam, Cambridge, MA, USA) and goat anti-rabbit HRP-conjugated secondary antibody at 1:5000 (#AB205718, abcam). H3Cit band intensities normalized to actin loading control were determined by ImageJ (NIH, Bethesda, MD).

### Mouse Neutrophils and Netosis Visualization

All mouse work was conducted under a protocol approved by Monash University's Animal Ethics Committee. Neutrophils were retrieved from peritoneal cavities of 4% thioglycollate-stimulated male C57BL/6 mice aged 10 weeks (N=8). Neutrophils plated at 2×10⁵ cells/well in 20-well plates were allowed to settle for 30 minutes and then treated with control media or media containing 10 μM DX1 for 30minutes. NETosis was stimulated by addition of PMA (40 μg/mL) or IM (4 μM, Stem Cell Technologies, VIC, Australia) for four hours. Cells were fixed in periodate-lysine-paraformaldehyde (PLP) overnight, immunostained for markers of NETs including myeloperoxidase (MPO, #AF3667 R&D Systems, MN, USA), H3Cit, and PAD4 (#AB128086, abcam), mounted in DAPI ProLong™ Gold (Molecular Probes, Thermo Fisher Scientific) and NETs were visualized by confocal fluorescence microscopy with a Nikon Ti-E inverted microscope (Nikon Instruments Inc, Melville, NY, USA). 405,488, and 561, and 647 nm lasers were used to specifically excite DAPI, Alexa 488, Alexa 594, and Alexa 647. The percentage of NETs formed in each treatment condition was determined using ImageJ (NIH).

### Statistics

Statistical analyses were performed in GraphPad Prism, version 9.2. P values were determined by two-tailed Student's t-test when comparing two groups and one-way ANOVA with Tukey's multiple comparisons test for multiple groups. P<0.05 was considered significant. Error bars in the figures represent standard error of the mean (SEM). Number of replicates are indicated in the figures.

### Mouse Neutrophils and Netosis Visualization

Neutrophils were retrieved from peritoneal cavities of 4% thioglycollate-stimulated mice (N=8) as previously described (24). Neutrophils plated at 2×10⁵ cells/well in 20-well plates were allowed to settle for 30 minutes and then treated with control media or media containing 10 μM DX1 for 30 minutes. NETosis was stimulated by addition of PMA (40 μg/mL) or ionomycin (IM) (4 μM, Stem Cell Technologies, VIC, Australia) for four hours. Cells were fixed in periodate-lysine-paraformaldehyde (PLP) overnight, immunostained for markers of NETs including myeloperoxidase (MPO, #AF3667 R&D Systems, MN, USA), H3Cit, and PAD4 (#AB128086, Abcam), mounted in DAPI Prolong Gold (Molecular Probes) and NETs were visualized by confocal microscopy (Nikon Ti-E inverted microscope, 405, 488, and 561 nm 647 nm lasers were used to specifically excite DAPI, Alexa 488, Alexa 594, and Alexa 647) The percentage of NETs formed in each treatment condition was determined using ImageJ (NIH).

### Mouse Model of ANCA Associated Vasculitis

As shown in FIG. 9, mice were immunised with MPO+FCA at day 0 and 7 followed by administration of anti-GBM and/or DX1 at day 16. Mice receiving two doses of DX1 received their second dose at day 18. Mice were culled for assessment at day 20.

Mice were separated into 4 groups: Ova control (n=4); MPO immunized no treatment with DX1 (n=8); MPO immunized and 1 dose of DX1 at 25 mg/kg (n=8) and; MPO immunized and 2 doses of DX1 at 25 mg/kg.

It will be appreciated by persons skilled in the art that numerous variations and/or modifications may be made to the disclosure as shown in the specific embodiments without departing from the spirit or scope of the disclosure as broadly described. The present embodiments are, therefore, to be considered in all respects as illustrative and not restrictive.

This application claims priority from U.S. Provisional Patent Application 63/238,657 filed on 30 Aug. 2021 and U.S. Provisonal Patent Application 63/324,784 filed on 29 Mar. 2022, the disclosures of which are incorporated herein in their entirety.

All publications discussed above are incorporated herein in their entirety. Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

1. A method of treating an inflammatory disease in a subject, the method comprising administering to the subject an effective amount of a cell penetrating, anti-DNA binding protein, wherein the cell penetrating, anti-DNA binding protein inhibits Neutrophil Extracellular Trap (NET) formation under culture conditions.

2. A method of inhibiting Neutrophil Extracellular Trap (NET) formation in a subject, the method comprising administering to the subject an effective amount of a cell penetrating, anti-DNA binding protein, wherein the cell penetrating, anti-DNA binding protein inhibits NET formation under culture conditions.

3. The method of claim 2, wherein the subject has an inflammatory disease.

4. The method according to any one of claims 1 to 3, wherein the cell penetrating, anti-DNA binding protein is an autoantibody derived from a subject or an animal with an autoimmune disease, preferably wherein the autoantibody is derived from a subject with systemic lupus erythematous, or an animal model thereof.

5. The method of claim 4, wherein the autoantibody is 3E10 or a humanised form thereof.

6. The method according to any one of claims 1 to 4, wherein the binding protein comprises a V_{H} having a CDR1 as shown in SEQ ID NO: 1, a CDR2 as shown in SEQ ID NO: 2, a CDR3 as shown in SEQ ID NO: 3 and a V_{L} having a CDR 1 as shown in SEQ ID NO: 4, a CDR2 as shown in SEQ ID NO: 5 and a CDR3 as shown in SEQ ID NO: 6 or a humanized form thereof.

7. The method of any one of claims 1 to 6, wherein the binding protein comprises a V_{H} which comprises an amino acid sequence as shown in SEQ ID NO: 7 and a V_{L} as shown in SEQ ID NO: 8 or a humanized form thereof.

8. The method of claim 6 or claim 7, wherein the humanized form thereof comprises a V_{H} having a CDR1 as shown in SEQ ID NO: 9 or SEQ ID NO: 10, a CDR2 as shown in SEQ ID NO: 11 or SEQ ID NO: 12, a CDR3 as shown in SEQ ID NO: 13 and a V_{L} having a CDR1 as shown in SEQ ID NO: 14 or SEQ ID NO: 15, a CDR2 as shown in SEQ ID NO: 16 and a CDR3 as shown in SEQ ID NO: 17 or a variant thereof that competes for binding to DNA with an antibody which comprises a V_{H} having a CDR1as shown in SEQ ID NO: 9 or SEQ ID NO: 10, a CDR2 as shown in SEQ ID NO: 11 or SEQ ID NO: 12, a CDR3 as shown in SEQ ID NO: 13 and a V_{L} having a CDR1 as shown in SEQ ID NO: 14 or SEQ ID NO: 15, a CDR2 as shown in SEQ ID NO: 16 and a CDR3 as shown in SEQ ID NO: 17.

9. The method of any one of claims 6 to 8, wherein the humanized form comprises a V_{H} which comprises an amino acid sequence as shown in any one of SEQ ID NOs: 18-21 and a V_{L} as shown in any one of SEQ ID NOs: 22-25 or a variant thereof that competes for binding to DNA with an antibody which comprises a V_{H} which comprises an amino acid sequence as shown in any one of SEQ ID NOs: 18-21 and a V_{L} as shown in any one of SEQ ID NOs: 22-25.

10. The method according to any one of claims 1 to 9, wherein the binding protein is nuclear penetrating.

11. The method according to claim 10, wherein the binding protein inhibits release of DNA from nuclei of neutrophils or neutrophil-like cells.

12. The method of any one of claims 1 to 11, wherein the binding protein is a di-scFv.

13. The method of any one of claims 1 to 12, wherein the binding protein comprises a linker which comprises an amino acid sequence shown in any one of SEQ ID NO: 17; SEQ ID NO: 26 or SEQ ID NO: 27.

14. The method of any one of claims 1 to 11, wherein the binding protein is an intact antibody.

15. The method according to any one of claims 1, 2 or 3 to 14, wherein the inflammatory disease is selected from the group consisting of cystic fibrosis, ischemia, cardiovascular disease, periodontitis, fibrosis, pruritus, skin inflammation, psoriasis, multiple sclerosis, rheumatoid arthritis, osteoarthritis, systemic lupus erythematosus, Hashimoto's thyroidis, myasthenia gravis, diabetes type I or II, diabetic nephropathy, asthma, inflammatory liver injury, inflammatory glomerular injury, atopic dermatitis, allergic contact dermatitis, irritant contact dermatitis, seborrhoeic dermatitis, Sjoegren's syndrome, keratoconjunctivitis, uveitis, vasculitis, inflammatory bowel disease, Crohn's disease, ulcerative colitis, acute or chronic idiopathic inflammatory arthritis, myositis, a demyelinating disease, chronic obstructive pulmonary disease, interstitial lung disease, interstitial nephritis, chronic active hepatitis, gout, metabolic disease, inflammation associated with obesity, septic arthritis, aseptic arthritis, disseminated intravascular coagulation (DIC), Alzheimer's disease.

16. The method according to any one of claims 1, 2 or 3 to 14, wherein the inflammatory disease is selected from the group consisting of pneumonia, neutrophilic asthma, neutrophil-mediated anaphylaxis, inflammatory lung injury, chronic obstructive pulmonary disease (COPD), or coronavirus disease 19 (COVID-19).

17. The method according to any one of claims 1, 2 or 3 to 14, wherein the inflammatory disease is vasculitis, preferably ANCA associated vasculitis.

18. The method according to any one of claims 1, 2 or 3 to 14, wherein the inflammatory disease is acute respiratory distress syndrome.

19. The method according to any one of claims 1, 2 or 3 to 14, wherein the subject has or is at risk of developing a thrombosis or an embolism.

20. The method of claim 19, wherein the thrombosis is a venous or arterial thrombosis.

21. The method of claim 19, wherein the embolism is a pulmonary embolism.

22. The method according to any one of claims 1, 2 or 3 to 14, wherein the inflammatory disease is Acute Respiratory Distress Syndrome (ARDS).

23. The method according to any one of claim 1, 2 or 3 to 14 or 22, wherein the inflammatory disease is caused by a viral infection such as a rhinovirus, an influenza virus, a respiratory syncytial virus (RSV) or a coronavirus, preferably, wherein the viral infection is caused by a coronavirus.

24. The method of claim 23, wherein the coronavirus is severe acute respiratory syndrome coronavirus (SARS-CoV), Middle East respiratory syndrome coronavirus (MERS-CoV) or severe acute respiratory syndrome coronavirus 2 (SARS-CoV-2).

25. The method according to any one of claims 1 to 24, wherein culture conditions comprise culturing neutrophil-like PLB-985 cells or neutrophils in culture medium which comprises an inflammatory stimulus.

26. The method of claim 25, wherein the inflammatory stimulus causes NOX-dependent NETosis.

27. The method of claim 25, wherein the inflammatory stimulus is phorbol 12-myristate 13-acetate (PMA) or lipopolysaccharide (LPS).

28. The method of claim 25, wherein the inflammatory stimulus causes NOX-independent NETosis.

29. The method of claim 25, wherein the inflammatory stimulus is a calcium ionophore ionomycin (IM).

30. The method according to any one of claims 25 to 29, wherein the inhibition of NET formation under culture conditions is determined based on one or both of:
1. level of cell death and extracellular DNA in NETs; and/or,
2. reduced DNA release from neutrophil-like cells after culture with the inflammatory stimulus.


SEQUENCE LISTING (40 sequences)

SEQ ID NO: 1 - protein, 5 aa, synthetic construct
  REGION 1..5: note = 3E10 VH CDR1
DYGMH

SEQ ID NO: 2 - protein, 18 aa, synthetic construct
  REGION 1..18: note = 3E10 VH CDR2
YISSGSSTIYYADTVKGR

SEQ ID NO: 3 - protein, 7 aa, synthetic construct
  REGION 1..7: note = 3E10 VH CDR3
RGLLLDY

SEQ ID NO: 4 - protein, 5 aa, synthetic construct
  REGION 1..5: note = 3E10 VL CDR1
SYMHW

SEQ ID NO: 5 - protein, 6 aa, synthetic construct
  REGION 1..6: note = 3E10 VL CDR2
ASYLES

SEQ ID NO: 6 - protein, 9 aa, synthetic construct
  REGION 1..9: note = 3E10 VL CDR3
QHSREFPWT

SEQ ID NO: 7 - protein, 116 aa, synthetic construct
  REGION 1..116: note = 3E10 VH
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 8 - protein, 117 aa, synthetic construct
  REGION 1..117: note = 3E10 VL
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAP

SEQ ID NO: 9 - protein, 10 aa, synthetic construct
  REGION 1..10: note = VH CDR1 of DX1/DX3
GFTFSNYGMH

SEQ ID NO: 10 - protein, 10 aa, synthetic construct
  REGION 1..10: note = VH CDR1 variant of DX3
GFTFSNYGMN

SEQ ID NO: 11 - protein, 17 aa, synthetic construct
  REGION 1..17: note = VH CDR2 of DX1/DX3
YISSGSSTIYYADSVKG

SEQ ID NO: 12 - protein, 17 aa, synthetic construct
  REGION 1..17: note = VH CDR2 variant of DX3
YISNTSSTIYYADARKG

SEQ ID NO: 13 - protein, 9 aa, synthetic construct
  REGION 1..9: note = VH CDR3 of DX1/DX3
ARRGLLLDY

SEQ ID NO: 14 - protein, 15 aa, synthetic construct
  REGION 1..15: note = VL CDR1 of DX1/DX3
RASKTVSTSSYSYMH

SEQ ID NO: 15 - protein, 15 aa, synthetic construct
  REGION 1..15: note = VL CDR1 variant of DX3
RASKTVSTSSYGYMH

SEQ ID NO: 16 - protein, 7 aa, synthetic construct
  REGION 1..7: note = VL CDR2 of DX1/DX3
YASYLES

SEQ ID NO: 17 - protein, 9 aa, synthetic construct
  REGION 1..9: note = VL CDR3 of DX1/DX3
QHSREFPWT

SEQ ID NO: 18 - protein, 116 aa, synthetic construct
  REGION 1..116: note = VH of DX1/DX3
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 19 - protein, 116 aa, synthetic construct
  REGION 1..116: note = VH of DX3 variant 1
EVQLAESGGGLVQPGGSLRLSCAASGFTFSNYGMHWVRQAPGKGLEWVSYISSGSSTIYYADSVKGRYTISRENAKNSAYLQMNSLRAEDTGMYYCARRGLLLDYWGQGSNINISS

SEQ ID NO: 20 - protein, 116 aa, synthetic construct
  REGION 1..116: note = VH of DX3 variant 2
EVQLAESGGGLVQPGGSLRLSCAASGFTFSNYGMHWVRQAPGKGLEWVSYISSGSSTIYYADSVKGRYTISRENAKNSLYLQMNSLRAEDTGMYYCARRGLLLDYWGQGSNVTVSS

SEQ ID NO: 21 - protein, 116 aa, synthetic construct
  REGION 1..116: note = VH of DX3 variant 3
EVQLAESGGGLVQPGGSLRLSCAASGFTFSNYGMNWVRQAPGKGLEWVSYISNTSSTIYYADARKGRYTISRENAKNSAYLQMNSLRAEDTGMYYCARRGLLLDYWGQGSNINISS

SEQ ID NO: 22 - protein, 111 aa, synthetic construct
  REGION 1..111: note = VL of DX1/DX3
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 23 - protein, 111 aa, synthetic construct
  REGION 1..111: note = VL of DX3 variant 1
DIQMTHSPASLSASLGDRATITCRASKTVSTSSYSYMHWYQQKPGQPPKLLIKYASYLESGVPSRFSGSGSGTDYTLQINSLQPEDAATYYCQHSREFPWTFGGGTKVEIK

SEQ ID NO: 24 - protein, 111 aa, synthetic construct
  REGION 1..111: note = VL of DX3 variant 2
DIQMTQSPASLSASLGDRATITCRASKTVSTSSYSYMHWYQQKPGQPPKLLIKYASYLESGVPSRFSGSGSGTDYTLQINSLQPEDAANYYCQHSREFPWTFGGGTKVEIK

SEQ ID NO: 25 - protein, 111 aa, synthetic construct
  REGION 1..111: note = VL of DX3 variant 3
DIQMTHSPASLSASLGDRATITCRASKTVSTSSYGYMHWYQQKPGQPPKLLIKYASYLESGVPSRFSGSGSGTDYTLQINSLQPEDAANYYCQHSREFPWTFGQGTKVEIK

SEQ ID NO: 26 - protein, 21 aa, synthetic construct
  REGION 1..21: note = Linker 1
TVDAAPGGGGSGGGGSGGGGS

SEQ ID NO: 27 - protein, 19 aa, synthetic construct
  REGION 1..19: note = Linker 2
ASTKGPSVFPLAPLESTGS

SEQ ID NO: 28 - protein, 15 aa, synthetic construct
  REGION 1..15: note = Hinge sequence
EPKSCDKTHTCPPCP

SEQ ID NO: 29 - protein, 19 aa, synthetic construct
  REGION 1..19: note = Signal sequence
MEFGLSWVFLVALFRGVQC

SEQ ID NO: 30 - protein, 120 aa, synthetic construct
  REGION 1..120: note = VH of 5C6
QLKLVESGGGLVKPGGSLKLSCAASGFTFSSYTMSWVRQTPAKRLEWVATISSGGGSTYYPDSVKGRFTISRDNARNTLYLQMSSLRSEDTAMYYCARRAYSKRGAMDYWGQGTSVTVSS

SEQ ID NO: 31 - protein, 5 aa, synthetic construct
  REGION 1..5: note = VH CDR1 of 5C6
SYTMS

SEQ ID NO: 32 - protein, 16 aa, synthetic construct
  REGION 1..16: note = VH CDR2 of 5C6
TISSGGGSTYYPDSVK

SEQ ID NO: 33 - protein, 11 aa, synthetic construct
  REGION 1..11: note = VH CDR3 of 5C6
RAYSKRGAMDY

SEQ ID NO: 34 - protein, 112 aa, synthetic construct
  REGION 1..112: note = VL of 5C6
DIVLTQSPASLAAVSLGERATISYRASKSVSTSGYSYMHWNQQKPGQAPRLLIYLVSNLESGVPARFSGSGSGTDFTLNIHPVEEEDAATYYCQHIRELDTFFGGGTKLEIK

SEQ ID NO: 35 - protein, 15 aa, synthetic construct
  REGION 1..15: note = VL CDR1 of 5C6
RASKSVSTSGYSYMH

SEQ ID NO: 36 - protein, 7 aa, synthetic construct
  REGION 1..7: note = VL CDR2 of 5C6
LVSNLES

SEQ ID NO: 37 - protein, 9 aa, synthetic construct
  REGION 1..9: note = VL CDR3 of 5C6
QHIRELDTF

SEQ ID NO: 38 - protein, 515 aa, synthetic construct
  REGION 1..515: note = DX1 sequence
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKTVDAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESTGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKTVDAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 39 - protein, 664 aa, synthetic construct
  REGION 1..664: note = DX3 sequence
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 5 aa, synthetic construct
  REGION 1..5: note = Alternate heavy chain CDR1 of 3E10
NYGMH